(19) United States
(12) Patent Application Publication
(10) Pub. No.: US 2005/0095253 A1
(43) Pub. Date: May 5, 2005

(54) Tumour associated antigen 791Tgp72

(76) Inventors: Linda Gillian Durrant, Nottingham (GB); Ian Spendlove, Nottingham (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/901,601

(22) Filed: Jul. 28, 2004

Related U.S. Application Data
(63) Continuation of application No. 09/623,035, filed on Oct. 12, 2000, now abandoned.

(30) Foreign Application Priority Data
Feb. 26, 1998 (GB) 9804065.2

(51) Int. Cl. A61K 39/00; C12P 21/04

(52) U.S. Cl. 424/185.1

(57) ABSTRACT

Cancer vaccines comprising a polypeptide of the CD55 family, or fragments or derivatives of polypeptides of the CD55 family. A preferred polypeptide is the 791Tgp72 antigen. Also provided are isolated and purified 791Tgp72 antigens.

### FIELD OF THE INVENTION

The present invention relates to tumour associated antigen 791Tgp72 and a method for its isolation, and to the use of 791Tgp72 and/or CD55 and related substances in methods of medical treatment, in particular as cancer vaccines.

### BACKGROUND OF THE INVENTION

A mouse monoclonal antibody 791T/36 was raised against the osteosarcoma cell line 791T (Embleton et al, 1981). A cell line expressing this antibody is deposited with the ATCC under accession number HB9173. Immunoprecipitation studies showed that 791T/36 recognised a membrane glycoprotein of molecular weight 72,000 (Price et al, 1984). A similar antigen can also be precipitated from activated human T lymphocytes. Extensive studies have shown that 791T/36 binds to the majority of osteosarcomas and also to colorectal, gastric and ovarian tumours (Durrant et al, 1986; Durrant et al, 1989; Durrant et al, 1989). The tumour specificity of 791Tgp72 was also emphasised by extensive clinical imaging studies with radiolabelled 791T/36 in the detection of primary and metastatic colorectal cancer, osteosarcoma, breast and ovarian cancer. The antibody was also liked to ricin A chain and showed good killing of tumour cells expressing the 791Tgp72 antigen. A phase I clinical study showed that the dose limiting toxicity was due to vascular leak syndrome and neurological toxicity of the ricin and was unrelated to antibody binding.

During the course of the clinical imaging and toxin targeting studies with 791T/36, it became clear that a limitation was the induction of human anti-mouse antibody responses (HAMA) (Durrant et al, 1989) which could limit the effectiveness of subsequent therapy with this monoclonal antibody. A large component of this HAMA response was directed at the idiotype of 791T/36. Most patients made a very strong anti-idiotypic response suggesting that a pre-existing helper T-cell response to tumour expressed 791Tgp72 antigen allowed preferential help for an anti-idiotypic response. Indeed a patient who had already survived 3 years with metastatic colon cancer received radiolabelled 791T/36 for tumour imaging. He made a very strong idiotypic response which resulted in anaphylactic shock suggesting that the pre-existing helper response to the 791Tgp72 may have been stabilising his disease and had been boosted with the injection of 791T/36. He recovered and lived a further 4 years finally succumbing to bone metastases. A human monoclonal anti-idiotypic antibody which bound to the antigen combining site of 791T/36 was produced from this patient (Austin-et al, 1989 and WO90/04415). Similarly immunisation of mice with 791T/36 linked to ricin induced a strong anti-idiotypic response and a mouse monoclonal anti-idiotypic antibody to 791T/36 was produced.

Clinical and laboratory studies with the human anti-idiotypic antibody have shown that it is an excellent immunogen for stimulating anti-tumour T-cell mediated immunity. 105AD7 can prime delayed hypersensitivity responses in rats and mice to human tumour cells expressing 791Tgp72 antigen. No toxicity related to anti-idiotypic immunisation has been observed in any of the 164 patients entered into phase I/II clinical trials with 105AD7. Patients in the phase I study showed T-cell proliferation responses to both the 105AD7 immunogen and also to the target antigen 791Tgp72 which correlated with survival. The lack of toxicity and excellent immune responses has enabled us to undertake a trial in primary colorectal cancer patients where evidence of autologous anti-tumour cytotoxicity was observed in patients immunised with 105AD7 prior to surgery. Single CTL epitope vaccines may not be very effective as some tumour cells lack the target antigen. This is less of a problem when stimulating helper T-cell responses due to different effector mechanisms. Antigen stimulation and homing occur by a similar mechanism to CTL. However, once at the tumour site, helper T-cells release cytokines which initiate a cascade of inflammatory events resulting in recruitment of effector cells which can kill tumour cells independent of their antigen status. This kind of infiltration profile has been seen in the tumours of patients following 105AD7 immunisation. CD4 and CD8 T-cells and natural killer cells were elevated in immunised patients compared to unimmunised. Furthermore, immunised patients had enhanced natural killer cell activity, which is of great significance as colorectal tumours often lose expression of MHC molecules resulting in susceptibility to NK killing.

### SUMMARY OF THE INVENTION

Previous attempts to purify and identify the 791Tgp72 antigen using both immunoprecipitation and affinity chromatography failed due to poor yields and the conformational dependence of 791T/36 for antigen binding. A modified method of affinity purification of 791Tgp72 has now been developed which has led to the isolation of this antigen for the first time. Biotinylation of cell membranes has allowed us to optimise the purification protocol, enabling efficient tracing of purified fractions. The use of the mild detergent octyl-glucoside and the introduction of an ultracentrifugation step has enhanced the purification 50-100 fold. The affinity chromatography has significantly been improved by covalently coupling the capturing antibody (791T/36) to protein-A sepharose. We have purified over 100 μg of the antigen and N-terminal sequencing has identified the molecule as being a member of the CD55/DAF family.

Further sequencing has revealed that the coding region of 791Tgp72 cDNA is the same as that for a known CD55 protein (herein known as “CD55”). Hence the amino acid sequences of 791Tgp72 and CD55 are also identical. There are, however, differences between the 791Tgp72 and CD55 proteins, for example in the glycosylation pattern of the molecules.

Accordingly, in a first aspect, the present invention provides isolated and purified 791Tgp72 antigen.

In a further aspect, the present invention provides isolated and purified 791Tgp72 antigen as obtainable by:

(a) solubilising 791T cells in lysis buffer including 1% octyl-B-glucoside, pH 8.5 for 1 hour at 4° C.;

(b) centrifuging the lysate at 13000 rpm ×10 min following 100,000 g ×30 min;

(c) adding the cleared lysate to Protein A sepharose coupled to 791T/36 affinity column;

(d) cycling the supernatant over the column at 0.3-0.4 ml/min;

(e) washing the column with 20 ml 20 mM Tris-HCl pH 8.0 containing 0.3 M NaCl and 0.1% NP-40; and,

(f) eluting 791Tgp72 from the column in 5 column volumes of diethylamine pH 11.5 containing 0.5% NP-40 and neutralising the eluate with 1M Tris.

In a further aspect, the present invention provides a pharmaceutical composition comprising 791Tgp72 in combination with a pharmaceutically acceptable carrier.

In a further aspect, the present invention provides 791Tgp72 for use in a method of medical treatment.

In a further aspect, the present invention provides a method for isolating 791Tgp72 antigen from cells expressing 791Tgp72, the method including the steps of:

solubilising the cells with lysis buffer including octyl-glucoside; and

treating the lysate using ultracentrifugation.

The inventors found that these steps surprisingly helped to enhance the purification of the antigen 50-100 fold.

The isolation and characterisation of 791Tgp72 carried out for the first time here identified this antigen as a member of the CD55 or decay accelerating factor (DAF) family. Thus, the use of 791Tgp72 as a cancer vaccine proposed herein can be extended to other CD55 polypeptides, a variety of forms of which have been isolated in the prior art, and to fragments and derivatives of these molecules. Likewise, the use of nucleic acid sequences encoding 791Tgp72 or its fragments and derivatives can be extended to nucleic acid sequences encoding other CD55 family members, and their fragments and derivatives.

Accordingly, in a further aspect, the present invention provides a cancer vaccine comprising 791Tgp72 antigen or a polypeptide of the CD55 family, or a fragment or derivative of T791Tgp72 or of a polypeptide of the CD55 family, wherein the vaccine is capable of inducing an immune response in a patient. The response may be one or more of a T-helper cell response, a cytotoxic T-cell response, an NK cell response and/or an immune response.

In a further aspect, the present invention provides a cancer vaccine comprising nucleic acid encoding 791Tgp72 and/or a polypeptide of the CD55 family, or a fragment or derivative of T791Tgp72 or of a polypeptide of the CD55 family, wherein the vaccine is capable of inducing an immune response in a patient. Again, the response may be one or more of a T-helper cell response, a cytotoxic T-cell response, an NK cell response and/or an immune response.

In a further aspect, the present invention provides the use of 791Tgp72 antigen or a polypeptide of the CD55 family, or a fragment or derivative of T791Tgp72 or of a polypeptide of the CD55 family, in the preparation of a medicament for the treatment of cancer.

In a further aspect, the present invention provides the use of nucleic acid encoding 791Tgp72 antigen or a polypeptide of the CD55 family, or a fragment or derivative of T791Tgp72 or of a polypeptide of the CD55 family, in the preparation of a medicament for the treatment of cancer.

In a further aspect, the present invention provides a method of treating a patient having cancer, the method comprising administering to the patient a therapeutically effective amount of one of the above cancer vaccines.

The use of 791Tgp72 to stimulate a T-cell response to cancer cells bearing 791Tgp72 is unexpected, as it is thought that the cancer cells have evolved to express these antigens to protect them from complement mediated attack. Thus, it is surprising that this defense mechanism of the tumour cells provides a way of selectively targeting a T-cell response to cancer cells expressing high levels of these antigens. By way of example, the vaccine can be used to treat colorectal cancer, osteosarcoma, breast and ovarian cancer, all of which are associated with 791Tgp72 overexpression.

While 791Tgp72 antigen and CD55 are known to be over-expressed on a wide range of solid tumours, they are also expressed on normal red blood cells, leukocytes, endothelial cells and surface epithelial cells. However, the T-cell response induced by employing a vaccine based on these polypeptides should be capable of discriminating between the low level of expression on normal cells and the high levels on tumour cells. This is based on the observation that the binding of 791T/36 to tumour cells shows higher affinity than binding to red blood cells from experiments in which the passage of red blood cells through tumours resulted in transfer of the monoclonal antibody to the tumour cells. Thus, this suggests that the T-cell response will be targeted to tumours and immune clearance is avoided.

Clinical studies with the human monoclonal antibody 105AD7 which mimics the colorectal tumour associated antigen 791Tgp72 have shown that immunised patients show a range of anti-tumour T-cell responses as exemplified by antigen specific proliferation responses, enhanced IL-2 production, induction of CD45RO cells, infiltration of CD4, CD8 and CD56 cells within the tumours of immunised patients, enhanced natural killer activity and autologous tumour killing which was unrelated to NK killing. As the 105AD7 antibody vaccine has now been given to 164 patients with no associated toxicity, vaccines based on 791Tgp72 or CD55 may share this property.

Results below supporting the use of polypeptides of the CD55 family in an analogous manner to 791Tgp72 include:

(a) Sequence identity of 791Tgp72 and CD55.

(b) Monoclonal antibodies specific to CD55 bind to purified 791Tgp72 antigen.

(c) 791T/36 binds to cells transfected with CD55.

(d) 791T/36 binds to cells transfected with CD55/C46 chimeric constructs which contain CD55 SUSHI domain 2.

(e) 791T/36 and monoclonal antibodies specific to CD55 immunoprecipitate two proteins of 72 and 66 kDa from the 791T osteosarcoma cell line. However, the yield of the dimer is far greater with 791T/36 than with the anti-CD55 monoclonal antibodies.

(f) 791T/36 and monoclonal antibodies specific to CD55 immunoprecipitate a single band of 72 kDa from normal red blood cells.

(g) 791T/36 recognises an epitope on 791Tgp72 as expressed by osteosarcoma cells, but binds weakly to red blood cells. In contrast, BRIC 216 (Blood Group Reference Laboratory, Bristol, UK), a monoclonal antibody which recognises CD55 as expressed by red blood cell, binds less well to osteosarcoma tumour cell lines as compared to 791T/36.

(h) 791Tgp72 is a GPI linked protein which is released by phospholipase C treatment.

(i) Radiolabelled 791T/36 localised within the ovarian and colorectal tumours and showed no detectable binding to red or white blood cells.

(j) 105AD7, an anti-idiotypic antibody which mimics 791Tgp72, has amino acid homology with the SCR2 domain (also known as SUSHI domain 2) of CD55.

(k) 730, an anti-idiotypic antibody which mimics 791Tgp72, has amino acid homology with the SCR2 domain of CD55.

(1) Ab3 responses induced by either the human or the mouse anti-idiotype may bind to CD55 on activated T-cells and enhance proliferation.

Furthermore, analysis of the amino acid sequence of CD55/791Tgp72 indicates that it may contain other T cell epitopes, which are distinct from the epitopes mimicked by 105AD7 and 730 anti-idiotypic antibodies. This suggests that vaccines comprising these other epitopes may induce immune responses in a broader range of patients than vaccines prepared from the anti-idiotype antibodies.

The present invention will now be described by way of example and not limitation, with reference to the accompanying figures.

### BRIEF DESCRIPTION OF THE FIGURES

FIG. 1 shows the detection of biotinylated proteins following SDS-PAGE and Western blotting. 791T cells were biotinylated and anti-DAF antibody (791T/36) or control antibody (1143/B7) was added either before or after solubilisation with 1% NP-40. The effect of crosslinking reagent (DTSSP) was assessed in the precipitation. X =Solubilisation after monoclonal antibody incubation with cells for 1 hour. 0 =Solubilisation of cells prior to addition of antibody.

FIG. 2 shows SDS-PAGE analysis of 791T/gp72 immunoprecipitates from cell surface biotinylated 791T cells. The samples were detected by the ECL reagent on Western blotted gels. The gel represents the effects of varying detergents and centrifugation protocols on sample purification.

FIG. 3 shows SDS-PAGE and silver staining of samples during protein purification. Lane 1, protein marker; (2) cell lysate; (3) unbound sample after passing the column; (4-7) samples from four consecutive 5 ml column washes; (8) concentrated washings; (9) samples of column eluate; and (10) concentrated column eluate. Each of the samples run on the gel was 25 μl volume. The washing and elution volumes were 5 ml.

FIG. 4 shows analysis of affinity purified 791T/gp72 by 7% SDS-PAGE and detected by silver staining. Lanes 1-5, 25 μl samples from consecutive 1.2 ml diethylamine eluates from the Protein-A affinity column. Lanes 6-10, varying concentrations of purified BSA.

FIG. 5 shows immunoprecipitation of cell surface biotinylated 791T cells by antibodies to DAF (110,BRIC 216) anti 791T/gp72 (791T/36) and anti EGF receptor monoclonal antibody (340). Experiments were carried out with the same amount of antibody, analysed by SDS-PAGE and western blotting and detecting using the ECL system. Lane 1 represents purified 791T/gp72. Lanes 4-7 represent precipitation with monoclonal antibodies 110, BRIC 216, 791T/36 and 340 respectively. Significantly more antigen is precipitated by 791T/36 compared to the anti-DAF antibodies.

FIG. 6 shows binding of anti-DAF antibodies (110,BRIC 216) and 791T/36 to affinity purified 791Tgp72 antigen and to PI-PLC released antigen from 791T cells.

FIG. 7 shows sandwich ELISA to determine if 791T/36 and the anti-DAF antibodies were binding distinct domains. Plates were coated with 791T/36, control antibody 708 (IgG2b) or anti-DAF antibodies; 220 (SUSHI domain 1), 110 (SUSHI domain 2), BRIC 216 (SUSHI domain 3). Binding of 791Tgp72 was detected with FITC-791T/36.

FIG. 8 shows non-reducing SDS-PAGE of proteins from erythrocyte and 791T cell membranes. Gels were Western blotted, cut into lanes, probed with appropriate antibody and developed using the ECL system.

FIG. 9 shows clustal alignment of CD55 and cloned products from 791T cells. 5/P5 represents 5 clones sequenced from the primer sequence P5. RC of B/C DAF represents 5 clones sequenced from the primers B DAF 3′ and c DAF 3′. CDAF.seq is the full length sequence of CD55 taken from GENBANK.

FIG. 10 shows the full length amino acid sequence of CD55 and the cDNA sequence which encodes it.

FIG. 11 shows the full length cDNA sequence of 791Tgp72 and the deduced amino acid sequence. 791Tgp72 has been found to have an identical amino acid sequence to CD55 and to be encoded by cDNA which is identical over the entire coding region to the cDNA encoding CD55, though differences exist in the 5′ and 3′ non-coding regions. These differences may be attributable to the use of different primers.

### DETAILED DESCRIPTION

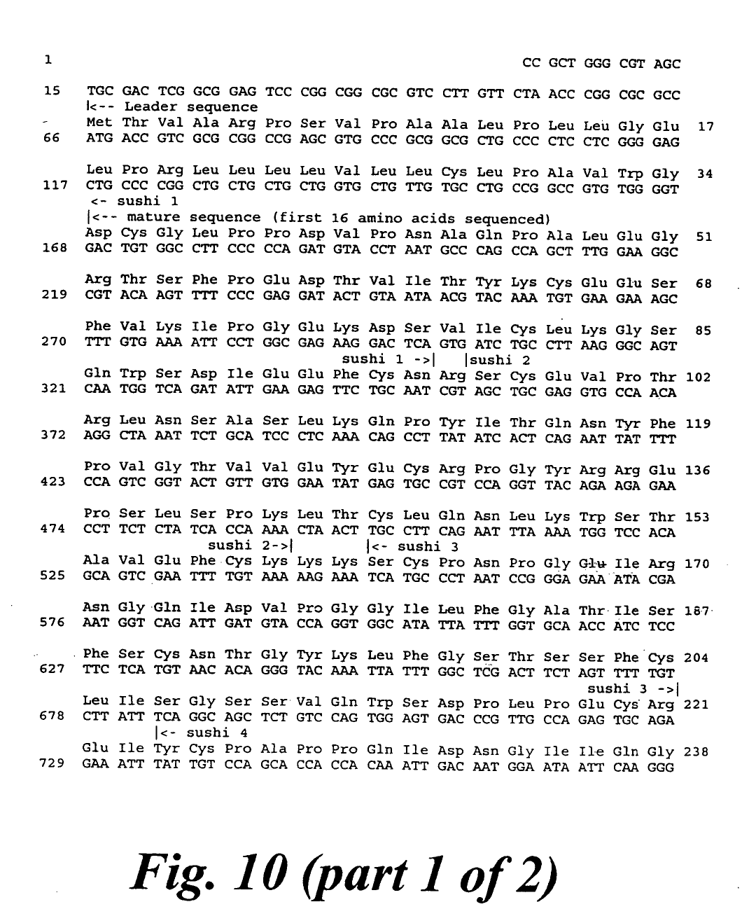

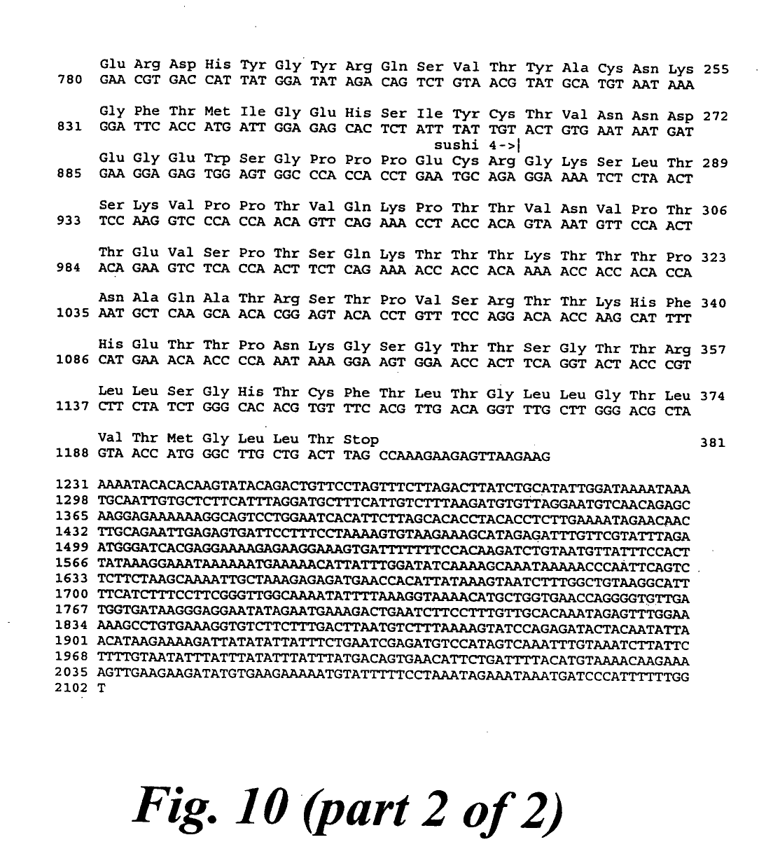

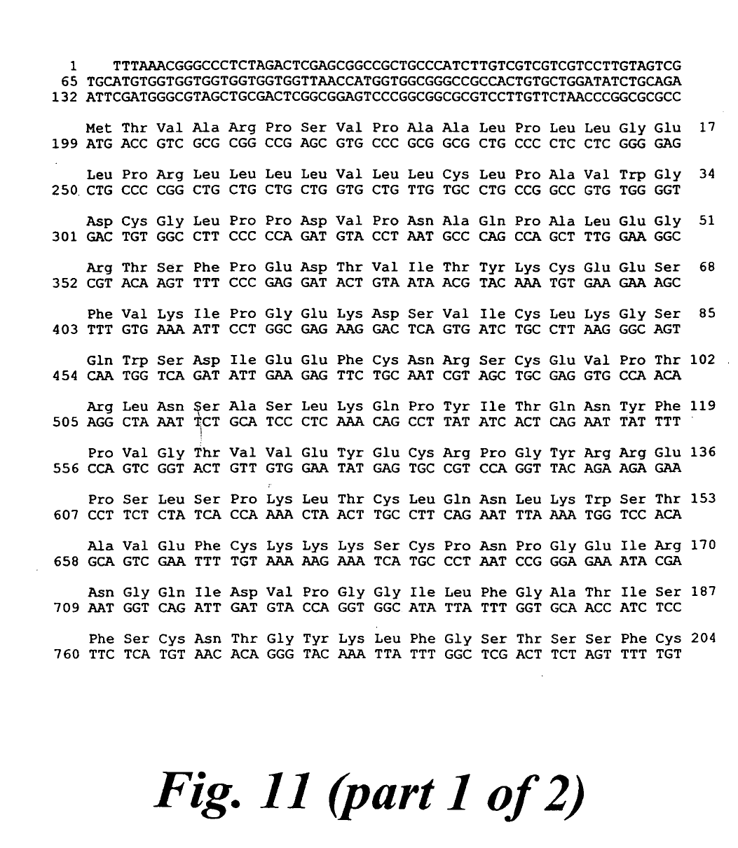

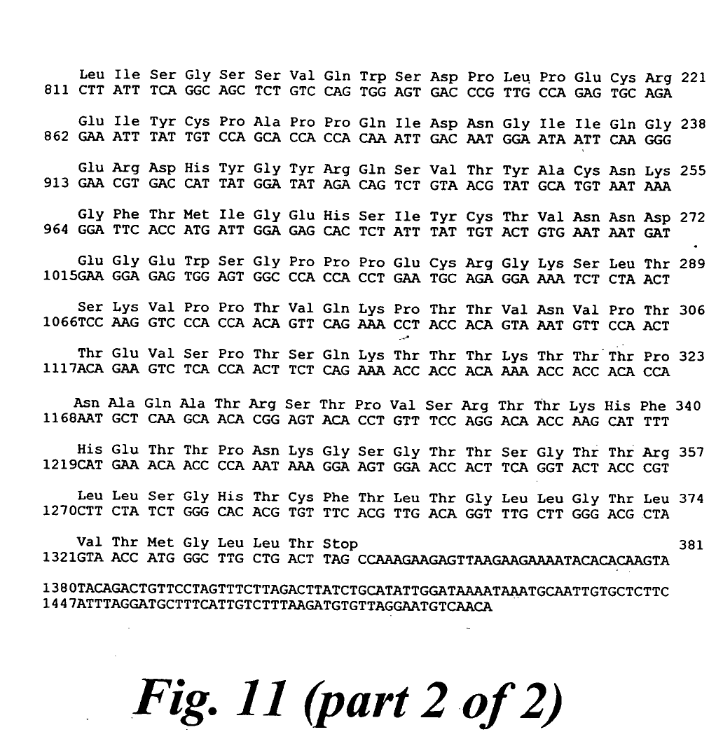

CD55, DAF and 791Tqp72 Polypeptides “791Tgp72 ” refers to the tumour associated antigen isolated in the work described herein from 791T cells that is bound by antibody 791T/36 (Embleton et al, 1981). This antigen is a member of the CD55 family, and has a high degree of amino acid homology with this known polypeptide. However, there are other differences between 791Tgp72 and other CD55 polypeptides, for example in the glycosylation pattern of the molecules. Further, different RNAs encoding 791Tgp72 antigen have been observed in the work described below and these may encode polypeptides having variations in amino acid sequence as compared to CD55. “CD55” refers particularly to the polypeptide having the sequence shown in FIG. 10. CD55 is also known as decay accelerating factor (DAF) and a variety of alternative forms of the polypeptide are known.

As used herein, “CD55 family” includes sequences which share substantial homology with CD55, such as the aforementioned alternative forms of the polypeptide (e.g. the previously identified CD55-A, CD55-B and CD55-U2), and which are capable of inducing in a patient an immune response against CD55 and/or 791Tgp72 as expressed on cancer cells. Preferably the degree of homology between CD55 and another protein of the CD55 family will be at least 60%, more preferably 70%, further preferably 80%, even more preferably 90%, or most preferably 95%.

CD55 was first purified by Nicholson-Weller et al from guinea pig and human erythrocytes (see Nicholson-Weller et al, 1981, 1982). Purified CD55 is a single chain glycoprotein with an Mr of 60,000 (guinea pig) or 70,000 (human) on SDS-PAGE. CD55 is initially synthesised as a precursor of 46 kDa, which gives rise to the mature CD55 on the cell surface with an MW of 70,000 to 80,000 due to heterogeneity in glycosylation. The structure of CD55 has been elucidated by a combination of biochemical studies and by the molecular cloning of cDNA. The cDNA for human CD55 encodes a 34-amino acid signal peptide followed by a 347-amino acid sequence of the protein. The amino terminus of the protein consists of four CCPR domains (also known as SUSHI or SCR domains). CD55 is anchored through covalent attachment to a GPI anchor.

As shown herein, antigen 791Tgp72 has an identical amino acid sequence to that of CD55 as shown in FIG. 10. The results described below suggest that CD55 and 791Tgp72, and fragments and derivatives thereof, can be used as cancer vaccines, to induce immune responses such as anti-tumour T-cell responses as exemplified by antigen specific proliferation responses, T-helper cell responses, cytotoxic T-cell responses, enhanced IL-2 production, induction of CD45RO cells, infiltration of CD4, CD8 and CD56 cells within the tumours of immunised patients, enhanced natural killer activity and/or autologous tumour killing which was unrelated to NK killing. Further, the peptides may act to raise CTL antibodies that neutralise CD55 and allow complement mediated lysis to take place.

Accordingly, the invention further includes the use of “fragments” or “derivatives” of either 791Tgp72 or other polypeptides of the CD55 family, which are less than the full length polypeptides, but which are capable of inducing an anti-tumour immune (especially T-cell) response as assessed by one or more of the indicators above. A preferred group of fragments are those which include all or part of the SUSHI2 domain of CD55 that stretches between amino acids 97-159 of full length CD55.

A “fragment” of a 791Tgp72 or of a polypeptide of the CD55 family means a stretch of amino acid residues of at least about five to seven contiguous amino acids, often at least about seven to nine contiguous amino acids, typically at least about nine to 13 contiguous amino acids, more preferably, at least about 20 to 30 or more contiguous amino acids, and most preferably at least about 30 to 40 or more consecutive amino acids.

A “derivative” of 791Tgp72 or of a polypeptide of the CD55 family, or of a fragment of 791Tgp72 or CD55 family polypeptide, means a polypeptide modified by varying the amino acid sequence of the protein, e.g. by manipulation of the nucleic acid encoding the protein or by altering the protein itself. Such derivatives of the natural amino acid sequence may involve insertion, addition, deletion and/or substitution of one or more amino acids, while providing a peptide capable of inducing an anti-tumour T-cell response.

Preferably such derivatives involve the insertion, addition, deletion and/or substitution of 25 or fewer amino acids, more preferably of 15 or fewer, even more preferably of 10 or fewer, more preferably still of 5 or fewer and most preferably of 1 or 2 amino acids only.

The invention also includes derivatives of the above peptides, including the peptide linked to a coupling partner, e.g. an effector molecule, a label, a drug, a toxin and/or a carrier or transport molecule. Techniques for coupling the peptides of the invention to both peptidyl and non-peptidyl coupling partners are well known in the art. In one embodiment, the carrier molecule is a 16 amino acid peptide derived from the homeodomain of Antennapedia (e.g. as sold under the name “Penetratin”), which can be coupled to a peptide via a terminal Cys residue. The “Penetratin” molecule and its properties are described in WO 91/18981.

Peptides may be generated wholly or partly by chemical synthesis. The compounds of the present invention can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, N.Y. (1984); and Applied Biosystems 430A Users Manual, ABI Inc., Foster City, Calif.), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Another convenient way of producing a peptidyl molecule according to the present invention (peptide or polypeptide) is to express nucleic acid encoding it, by use of nucleic acid in an expression system.

Accordingly the present invention also provides in various aspects nucleic acid encoding the polypeptides and peptides of the invention.

Generally, nucleic acid according to the present invention is provided as an isolate, in isolated and/or purified form, or free or substantially free of material with which it is naturally associated, such as free or substantially free of nucleic acid flanking the gene in the human genome, except possibly one or more regulatory sequence(s) for expression. Nucleic acid may be wholly or partially synthetic and may include genomic DNA, cDNA or RNA. Where nucleic acid according to the invention includes RNA, reference to the sequence shown should be construed as reference to the RNA equivalent, with U substituted for T.

Nucleic acid sequences encoding a polypeptide or peptide in accordance with the present invention can be readily prepared by the skilled person using the information and references contained herein and techniques known in the art (for example, see Sambrook, Fritsch and Maniatis, “Molecular Cloning, A Laboratory Manual, Cold Spring Harbor Laboratory Press, 1989, and Ausubel et al, Short Protocols in Molecular Biology, John Wiley and Sons, 1992), given the nucleic acid sequence and clones available. These techniques include (i) the use of the polymerase chain reaction (PCR) to amplify samples of such nucleic acid, e.g. from genomic sources, (ii) chemical synthesis, or (iii) preparing cDNA sequences. DNA encoding 791Tgp72 or CD55 fragments may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Modifications to the sequences can be made, e.g. using site directed mutagenesis, to lead to the expression of modified peptide or to take account of codon preference in the host cells used to express the nucleic acid.

In order to obtain expression of the nucleic acid sequences, the sequences can be incorporated in a vector having one or more control sequences operably linked to the nucleic acid to control its expression. The vectors may include other sequences such as promoters or enhancers to drive the expression of the inserted nucleic acid, nucleic acid sequences so that the polypeptide or peptide is produced as a fusion and/or nucleic acid encoding secretion signals so that the polypeptide produced in the host cell is secreted from the cell. Polypeptide can then be obtained by transforming the vectors into host cells in which the vector is functional, culturing the host cells so that the polypeptide is produced and recovering the polypeptide from the host cells or the surrounding medium. Prokaryotic and eukaryotic cells are used for this purpose in the art, including strains of E. coli, yeast, and eukaryotic cells such as COS or CHO cells.

Thus, the present invention also encompasses a method of making a polypeptide or peptide (as disclosed), the method including expression from nucleic acid encoding the polypeptide or peptide (generally nucleic acid according to the invention). This may conveniently be achieved by growing a host cell in culture, containing such a vector, under appropriate conditions which cause or allow expression of the polypeptide. Polypeptides and peptides may also be expressed in in vitro systems, such as reticulocyte lysate.

Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others. A common, preferred bacterial host is E. coli.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Thus, a further aspect of the present invention provides a host cell containing heterologous nucleic acid as disclosed herein.

The nucleic acid of the invention may be integrated into the genome (e.g. chromosome) of the host cell. Integration may be promoted by inclusion of sequences which promote recombination with the genome, in accordance with standard techniques. The nucleic acid may be on an extra-chromosomal vector within the cell, or otherwise identifiably heterologous or foreign to the cell.

A still further aspect provides a method which includes introducing the nucleic acid into a host cell. The introduction, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. As an alternative, direct injection of the nucleic acid could be employed.

Marker genes such as antibiotic resistance or sensitivity genes may be used in identifying clones containing nucleic acid of interest, as is well known in the art.

The introduction may be followed by causing or allowing expression from the nucleic acid, e.g. by culturing host cells (which may include cells actually transformed although more likely the cells will be descendants of the transformed cells) under conditions for expression of the gene, so that the encoded polypeptide (or peptide) is produced. If the polypeptide is expressed coupled to an appropriate signal leader peptide it may be secreted from the cell into the culture medium. Following production by expression, a polypeptide or peptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers (e.g. see below).

Pharmaceutical Formulations

The polypeptides, derivatives and fragments of the invention can be formulated in pharmaceutical compositions, and especially as vaccine compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes. The formulation is preferably liquid, and is ordinarily a physiologic salt solution containing non-phosphate buffer at pH 6.8-7.6, or may be lyophilized powder.

The compositions comprising or for the delivery of the 791Tgp72 and/or CD55 polypeptides are preferably administered to an individual in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. The vaccines of the invention are particularly relevant to the treatment of existing cancer and in the prevention of the reoccurrence of cancer after initial treatment or surgery. Examples of the techniques and protocols mentioned above can be found in Remington's Pharmaceutical Sciences, 16th edition, Oslo, A. (ed), 1980.

791Tgp72 antigen and/or polypeptides of the CD55 family, and/or their fragments and/or derivatives are prepared for administration by mixing them at the desired degree of purity with adjuvants or physiologically acceptable carriers, i.e. carriers which are non toxic to recipients at the dosages and concentrations employed. Adjuvants and carriers are substances that in themselves share no immune epitopes with the target antigen, but which stimulate the immune response to the target antigen. Ordinarily, this will entail combining active ingredient with buffers, low molecular weight (less than about 10 residues) polypeptides, proteins, amino acids, carbohydrates including glucose or dextrans, chelating agents such as EDTA, and other excipients. Freunds adjuvant (a mineral oil emulsion) has commonly been used for this purpose, as have a variety of toxic microbial substances such as mycobacterial extracts and cytokines such as tumour necrosis factor and interferon gamma. Other adjuvants for vaccination are disclosed in EP-A-0745388, W097/01330 and EP-A-0781559. Carriers can also act as adjuvants, but are generally distinguished from adjuvants in that carriers comprise water insoluble macromolecular particulate structures which aggregate the antigen, typical carriers include aluminum hydroxide, latex particles, bentonite and liposomes.

A composition may be administered alone or in combination with other treatments, either simultaneously or sequentially dependent upon the condition to be treated. Other cancer treatments include the 105AD7 antibody mentioned above, other chemotherapeutic agents, other radiotherapy techniques or other cancer vaccines known in the art. One particular application of the compositions of the invention are as an adjunct to surgery, i.e. to help to reduce the risk of cancer reoccurring after a tumour is removed.

It is envisioned that injections (intramuscular or subcutaneous) will be the primary route for therapeutic administration of the vaccines of this invention, intravenous delivery, or delivery through catheter or other surgical tubing is also used. Liquid formulations may be utilized after reconstitution from power formulations.

The polypeptide may also be administered via microspheres, liposomes, other microparticulate delivery systems or sustained release formulations placed in certain tissues including blood. Suitable examples of sustained release carriers include semipermeable polymer matrices in the form of shaped articles, e.g. suppositories, or microcapsules. Implantable or microcapsular sustained release matrices include polylactides (U.S. Pat. No:3,773,919, EP-A-0058481) copolymers of L-glutamic acid and gamma ethyl-L-glutamate (Sidman et al, Biopolymers 22(1): 547-556, 1985), poly(2-hydroxyethyl-methacrylate) or ethylene vinyl acetate (Langer et al, J. Biomed. Mater. Res. 15:16-277, 1981, and Langer, Chem. Tech. 12:98-105, 1982). Liposomes containing the polypeptides are prepared by well-known methods: DE 3,218,121A; Epstein et al, PNAS USA, 82:3688-3692, 1985; Hwang et al, PNAS USA, 77:4030-4034, 1980; EP-A-0052522; E-A-0036676; EP-A-0088046; EP-A-0143949; EP-A-0142541; JP-A-83-11808; U.S. Pat. Nos. 4,485,045 and 4,544,545. Ordinarily, the liposomes are of the small (about 200-800 Angstroms) unilamellar type in which the lipid content is greater than about 30 mol. % cholesterol, the selected proportion being adjusted for the optimal rate of the polypeptide leakage The 791Tgp72 and/or peptides of the CD55 family may be administered in a localised manner to a tumour site or other desired site or may be delivered in a manner in which it targets tumour or other cells.

Targeting therapies may be used to deliver the active agent more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

Instead of administering these agents directly, they may be produced in the target cells by expression from an encoding gene introduced into the cells, e.g. in a viral vector (a variant of the VDEPT technique - see below). The vector may targeted to the specific cells to be treated, or it may contain regulatory elements which are switched on more or less selectively by the target cells.

The agent may be administered in a precursor form, for conversion to the active form by an activating agent produced in, or targeted to, the cells to be treated. This type of approach is sometimes known as ADEPT or VDEPT, the former involving targeting the activating agent to the cells by conjugation to a cell-specific antibody, while the latter involves producing the activating agent, e.g. an enzyme, in a vector by expression from encoding DNA in a viral vector (see for example, EP-A-415731 and WO 90/07936).

Vectors such as viral vectors have been used in the prior art to introduce nucleic acid into a wide variety of different target cells. Typically the vectors are exposed to the target cells so that transfection can take place in a sufficient proportion of the cells to provide a useful therapeutic or prophylactic effect from the expression of the desired polypeptide.

A variety of vectors, both viral vectors and plasmid vectors, are known in the art, see U.S. Pat. No. 5,252,479 and WO 93/07282. In particular, a number of viruses have been used as gene transfer vectors, including papovaviruses, such as SV40, vaccinia virus, herpesviruses, including HSV and EBV, and retroviruses. Many protocols in the prior art have used disabled murine retroviruses.

As an alternative to the use of viral vectors other known methods of introducing nucleic acid into cells includes electroporation, calcium phosphate co-precipitation, mechanical techniques such as microinjection, transfer mediated by liposomes and direct DNA uptake and receptor-mediated DNA transfer.

Receptor-mediated gene transfer, in which the nucleic acid is linked to a protein ligand via polylysine, with the ligand being specific for a receptor present on the surface of the target cells, is an example of a technique for specifically targeting nucleic acid to particular cells.

The vaccination dose of the 791Tgp72 or CD55 family polypeptide will be dependent upon the properties of the vaccine employed, e.g. its binding activity and in vivo plasma half-life, the concentration of the polypeptide in the formulation, the administration route, the site and rate of dosage, the clinical tolerance of the patient involved, the pathological condition afflicting the patient and the like, as is well within the skill of the physician. For example, doses of 300 μg of polypeptide per patient per administration are preferred, although dosages may range from about 10 μg to 1 mg per dose. Different dosages are utilized during a series of sequential inoculations; the practitioner may administer an initial inoculation and then boost with relatively smaller doses of vaccine.

The vaccine compositions of the invention can be administered in a variety of ways and to different classes of recipients. Examples of types of cancer that can be treated with the vaccine include colorectal cancer, osteosarcoma, breast and ovarian cancers.

This invention is also directed to optimized immunization schedules for enhancing a protective immune response against cancer. By way of example, at least three separate inoculations with 791Tgp72 and/or CD55 family polypeptides be administered, with a second inoculation being administered more than two, preferably three to eight, and more preferably approximately four weeks following the first inoculation. It is preferred that a third inoculation be administered several months later than the second “boost” inoculation, preferably at least more than five months following the first inoculation, more preferably six months to two years following the first inoculation, and even more preferably eight months to one year following the first inoculation. Periodic inoculations beyond the third are also desirable to enhance the patient's “immune memory”. See Anderson et al, J Infectious Diseases 160 (6):960-969, December 1989 and the references therein. Generally, infrequent immunizations with polypeptides spaced at-relatively long intervals is more preferred than frequent immunizations in eliciting maximum antibody responses, and in eliciting a protective effect.

The above discussion, insofar as it relates to vaccine compositions and to the production of nucleic acid compositions, is generally applicable also to the nucleic acid vaccines of the present invention, in accordance with the following comments which relate specifically to such vaccines.

Nucleic acid immunisation involves the use of a nucleic acid, usually DNA, vector encoding a gene of interest. A preferred vector is pcDNA3 (Invitrogen, Groningen, Netherlands). A DNA sequence encoding the gene of interest is typically placed under the control of a eukaryotic promoter that allows for expression in the target mammalian cells. By inclusion of various known sequence tags the encoded gene product may be directed to various compartments within the cell. This may be used to influence the direction of the developing immune response, for example favouring CTL or antibody responses.

The vector is introduced into the mammalian body by a number of possible routes. For example, injection of a naked DNA vector into muscle or via an intradermal route has been successful in establishing immune responses, a typical protocol involving the intramuscular injection of 50 μg DNA into two muscles on three occasions. Other possible routes include encapsulation of the nucleic acid vector into particles that are taken up by antigen-presenting cells. Poly(lactide-coglycolide) PLG microparticles have been successfully used to raise immune responses by feeding the particles to mice.

A major advantage of nucleic acid immunisation is the prolonged production of immunogen from within the cells of the immunised mammal, in a similar way to that of viral infections. The vector nucleic acid has also been shown to be a stimulator of innate immunity, providing the right environment in which to establish an efficient and sustained immune response.

### Example 1

### Identification of 791Tgp72 Antigen by Inmunoprecipitation

To improve the yield of 791Tgp72 antigen, both immunoprecipitation and affinity chromatography biotinylation of cell membranes was used to optimise the purification protocol, enabling efficient tracing of purified fractions. Cell culture and surface biotinylation were carried out as described in Altin et al, 1994. The cell line 791T was cultured in RPMI 1640 medium supplemented with heat-inactivate 10% fetal calf serum (FCS). The cells were harvested with trypsin/EDTA and washed three times with ice-cold PBS-C/M before reacting with 0.5 mg/ml sulfo-NHS-biotin (Pierce) for 30 minutes at 4° C. In some experiments, biotinylation was carried out in the presence of the chemical cross-linking agent 3, 3-dithio-bis(sulfo-succinimidyl-propionate) (DTSSP; Pierce) to covalently link associated molecules. For these studies, cells were suspended in phosphate buffered saline minus CaCl₂/MgCl₂ (PBS-C/M, pH 7.6), biotinylated and then crosslinked for 1 hr at room temperature with gentle mixing, following the manufacturers recommendations (PIERCE). Initial precipitations were carried out on biotinylated samples. Antibody (791T/36) was added to either whole cells or cell lysates. For these experiments 1143/B7 Mab was used as the negative control antibody.

Cells (2-5 ×10⁷) were lysed for 2 hrs at 4° C., cell lysates were cleared by centrifugation at 13000 rpm for 15 minutes. Immune complexes were then formed with protein-A sepharose (Sigma) for 30 minutes at 4° C. This basic protocol allowed us to vary the detergents and their concentrations, washing conditions and incubation times in order to optimise the purification protocol. Detergents tested were 0.5, 1.0 and 1.5%; Nonidet P-40, Tween-20 and Octyl Glucoside. These were used in TNE (20mM Tris, pH 8.0, 140 mM NaCl, 5mM EDTA). Washes were carried out with (20mM Tris pH 8.0, 100 mM NaCl, lmM EDTA, 0.1 mM PMSF +0.25% detergent). Washed protein-A sepharose beads were boiled in sample buffer (+/−) mercaptoethanol, reducing or non-reducing conditions. The samples were analysed by SDS-PAGE, Western blotted onto nitrocellulose membrane (Hybond™-C; Amersham), and detection of biotinylated proteins were carried out as described in Laemmli (1970), Stern (1993) and Dunbar (1994).

After transferring, the membrane was briefly washed with PBS and dried for 30 minutes at room temperature before blocking with PBS containing 0.1% Tween-20 and 1% BSA. The membrane was then washed twice for 5 minutes with PBS containing 0.1% Tween-20 and then incubated with horseradish peroxidase (HRPO)-streptavidin (1:1500; GIBCOBRL) for 1 hour at room temperature. The membrane was then washed three times (as above) and proteins were detected using the enhanced chemiluminescence (ECL) protein detection system (Amersham) by exposing the chemiluminescent blot to X-OGRAPH film. The detection:-of non-biotinylation proteins was carried out by silver staining.

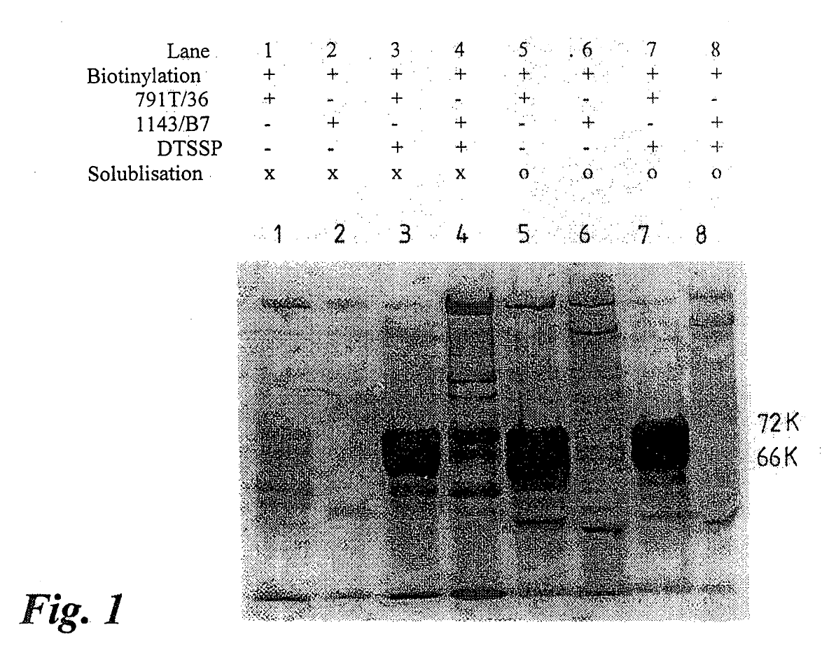

FIG. 1 shows the results of immunoprecipitation by mAb 791/36 from 791T cells. All cells were solublised in TNEN buffer containing 1% NP-40. The binding reaction was carried out before (Lanes 1-4) or after (Lanes 5-8) cell solubilisation. Cross linking reagent (DTSSP) was used in some of the reactions (Lanes 3, 4, 7 and 8) and the precipitation was carried out using 79T/36 (odd lane numbers) or 1143/B7 control antibody (even lane numbers). It can be seen that crosslinking the antibody to the cell surface (Lane 3) improved the amount of purified antigen compared to cells solublised without crosslinking. Comparable results were obtained with cells solublised prior to antibody addition (lanes 5 and 7).

### Example 2

### Checking the Conditions for Purification of the Antigen

In order to optimise the conditions for the purification of large amounts of 791Tgp72 the conditions were varied as for purification of the biotinylated protein. Initially, CNBr-activated sepharose 4B was used to make an affinity column with 791T/36 Mab (see Hole et al, 1988; Hole et al, 1990; Goding, 1996), but this proved very inefficient. A modification of this procedure using Protein A sepharose was introduced (Scneider et al, 1982). 1-2 ×10⁹ 791T cells were solubilized in 100 ml of 1% octyl glucoside in Tris buffer (20 mM Tris-HCl pH 8.5, 150 mM NaCl, 25 mM benzamidine, 5 mM EGTA, 10 ug /ml leupeptin, 0.1 mM PMSF) for 1 hr at 4° C. with continual mixing. Unsolubilized material was discarded after centrifugation at 13000 rpm for 10 minutes, this was followed by a 100,000 g centrifugation of the supernatant for 30 minutes. The solubilized material was loaded on to the protein-A Sepharose-791T/36 crosslinked affinity column with a flow rate of 0.3-0.4 ml/min. The column was then washed with 20 ml 50 mM Tris-HCl pH 8.0 containing 0.3 M NaCl with 0.1% NP-40. The 791Tgp72 antigen was eluted with 5 column volume of 50 mM diethylamine pH 11.5 containing 0.5% NP-40. The sample was immediately neutralised by adding 200 ul of 1 M Tris-HCl pH 8.0. The original sample was recycled over the column another 2-3 times as above to recapture any unpurified antigen. Fractions were assessed by SDS-PAGE and silver staining.

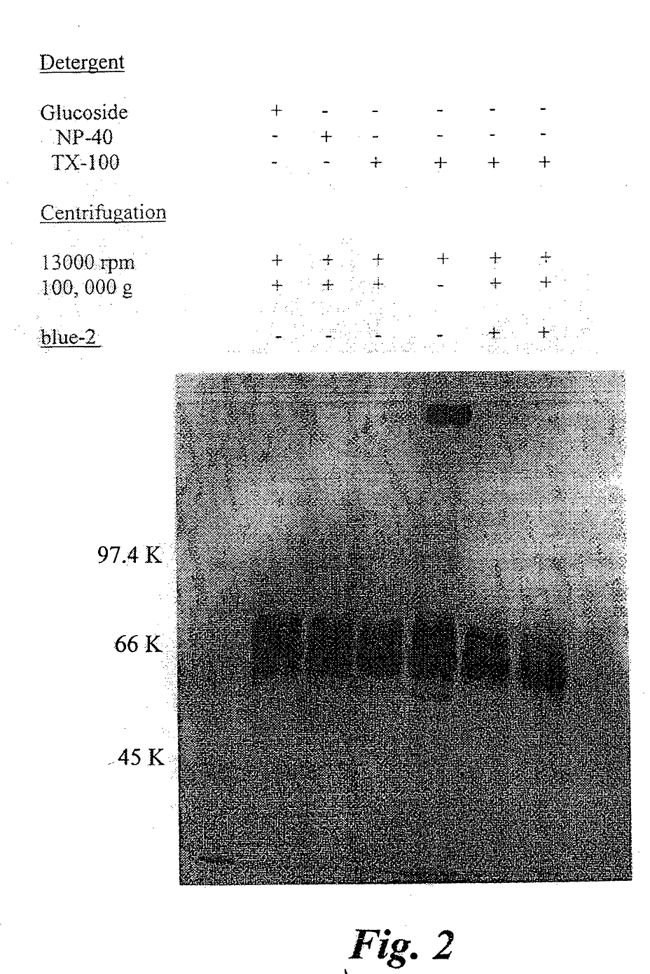

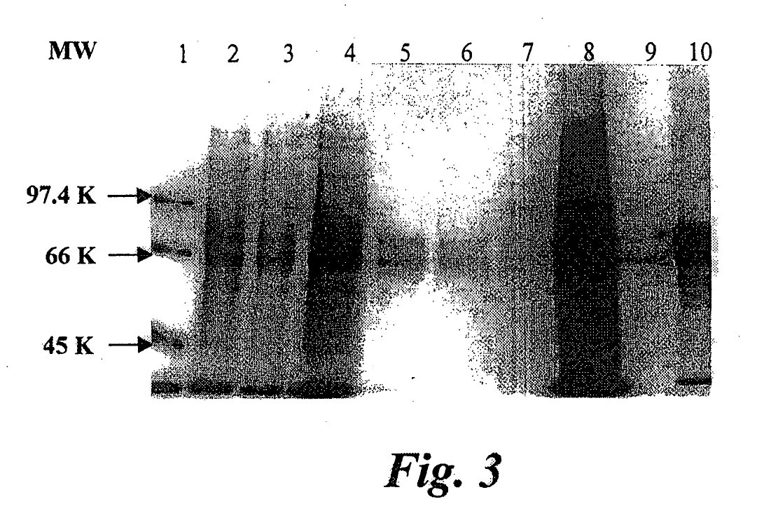

FIG. 2 shows the effects of various conditions on purification efficiency. Lane 2-4 represent three cell lysates solubilized by different non-ionic detergents. Octyl-B-glucoside yielded more precipitates (lane 2) and ultracentrifugation when used achieved significant improvements in reducing background protein contamination. Using the 791T36-protein-A sepharose column and similar conditions we were able to show significant improvements in yields of antigen (FIG. 3, lane 10). However, we also showed that antigen was also eluted by even the mildest washing conditions (FIG. 3, lane 3-9)

Following analysis of the purification procedures, the final conditions were chosen for affinity chromatography:
(1) Lysis buffer with 1% octyl-B-glucoside, pH 8.5 for 1 hour at 4° C.
(2) Lysate centrifugation: 13000 rpm ×10 min following 100,000 g ×30 min.
(3) Addition of cleared lysate to Protein A sepharose coupled to 791T/36 affinity column.
(4) Cycle the supernatant over the column at 0.3-0.4 ml/min.
(5) Washing the column with 20ml 20 mM Tris-HCl pH 8.0 containing 0.3 M NaCl and 0.1% NP-40.
(6) Sample was eluted in 5 column volumes of diethylamine pH 11.5 containing 0.5% NP-40 and neutralised with 1M Tris.
(7) The sample solution was recycled for 3-4 times to recover as much antigen as possible.

### Example 3

### Sequence Analysis

To determine the N-terminal amino acid sequence, affinity purified 791Tgp72/66 was concentrated using vivaspin centrifugation columns. Approximately 10 μg of protein was analysed by SDS-PAGE and Western blotted onto PVDF membrane (PROBLOT, ABI) following the manufacturer's recommendations, with a modification by addition of 0.1% SDS. Following transfer for 1-2 hrs, the blot was stained with Coomassie blue for 30 seconds and rinsed in 10% methanol 20% acetic acid. The stained 66 and 72 KDa bands were excised from the blot and subjected to 16 rounds of automated protein sequencing on an ABI XXX sequencer.

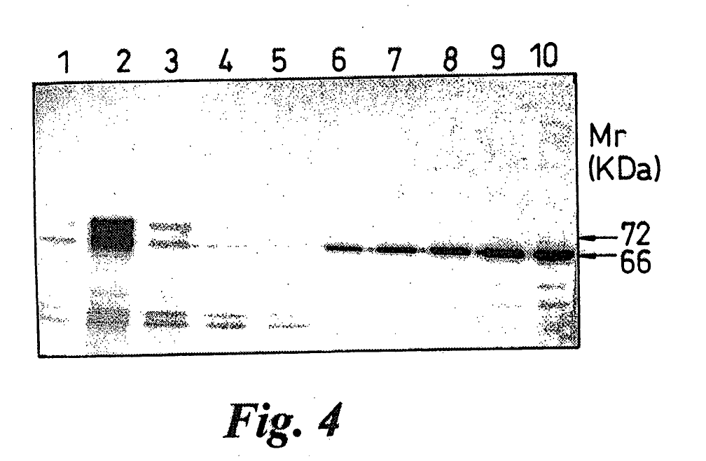

FIG. 4 shows the results of silver staining from the fractions of protein A column. The antigens of 791Tgp72 and p66 were eluted in 2-3 fractions.

N-terminal sequence analysis gave the following sequence “DCGLPPDVPNAQPALE” which showed 100% identity with the sequence of decay accelerating factor (DAF, CD55).

### Example 4

### Transfection of CD55 into CHO Cells

To check the recognition of CD55 by 791T/36 Mab, CHO cells were transfected with a CD55 cDNA clone. The clone was obtained from Dr Dale Christiansen (Austin Research Institute, Victoria 3084, Australia). Cells transfected with the clone were assayed by FACS analysis for binding of anti-CD55 antibodies, 110 and BRIC 216 and also for binding of 791T/36. All the antibodies showe good binding to the CHO cells transfected with CD55 but no binding to untransfected cells, see Table 1.

### Example 5

### Flow Cytometry Anti-CD55 Binding Assay

To measure expression of 791Tgp72 and CD55 on tumour and primary cell lines, 2×10⁵ of 791T, human umbilical vein endothelial cells (HUVEC) and erythrocytes were mixed with cold anti-CD55 110, BRIC 216, 220, 791T/36 and control mAb 708 (0.1 pg) separately at 37° C. for 1 hr. Then rabbit anti-mouse FITC (1:100, DAKO, Denmark) was added to each tube and incubated for another 1 hr. Direct binding of 791T/36 FITC (0.1 Ag) to 791T cells was measured after 1 hr at 37° C. The cells were washed two times with RPMI 1640 medium, fixed and measured by flow cytofluorometry.

Table 2 present the results of antibodies binding to different cell lines. The data show that both anti-CD55 and 791T/36 mabs bind to red blood cells, HUVEC cells and to the osteosarcoma cell line 791T. The anti-CD55 antibody BRIC 216 bound most strongly to red blood cells and HUVEC cells whereas 791T/36 showed the strongest binding to 791T cells which was approximately 2 orders-of magnitude higher than to the normal cells. These results suggest that 791Tgp72 is closely related to CD55 but that there are some differences. These differences could be differential glycosylation or post-translational modifications, e.g. point mutations.

### Example 6

### Immnunoprecipitation with Various Anti-CD55 Antibodies

To confirm whether anti-CD55 monoclonal antibodies could precipitate an antigen from tumour cells, the same immunoprecipitation protocol as mentioned previously was used. 40 μg of anti-CD55 110, BRIC 216 and anti-791Tgp72, 91T/36 were used to precipitate the antigen from 2×10⁷ 791T cells respectively.

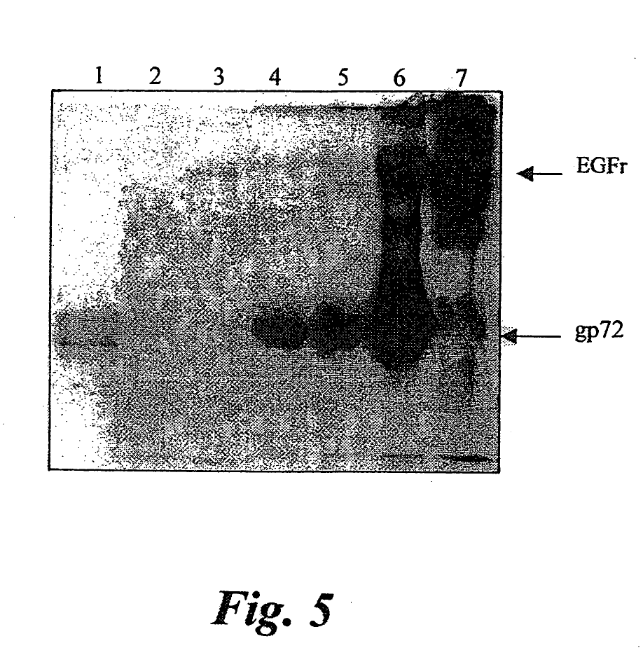

Both anti-CD55 monoclonal antibodies, 110, BRIC 216 and 791T36 immunoprecipitated an antigen of similar molecular weight although the yield was far greater with 791T/36 than the with the anti-CD55 antibodies (FIG. 5). These results again suggest that a similar antigen is precipitated by both the anti-CD55 antibodies and 791T/36, but that the later Mab has either better access or a higher affinity for 791Tgp72.

### Example 7

### Phosphatidylinositol Phospholipase C (PI-PLC) Treatment

CD55 is a GPI linked protein. To confirm whether 791Tgp72 is also GPI linked, 791T cells were treated with Phosphatidylinositol phospholipase C (PI-PLC; Boehringer Mannheim, Germany), to release GPI linked antigens. Cells (5×105) were incubated with PI-PLC(1 U/ml) for 1 hour at 37° C. The cells were washed two times with PBS and the expression of CD55 and or 791Tgp72 was determined by indirect immunofluorescence binding with monoclonal antibodies and flow cytometric analysis.

As shown in Table 3, the binding of anti-CD55 monoclonal antibodies and 791T/36 decreased after incubation with PI-PLC for 1 hr, with a maximal decrease in surface expression of approximately 85-90%. Cells incubated in parallel without PI-PLC retained their surface expression of 791Tgp72 antigen. These results clearly show that 791Tgp72 is also GPI linked.

### Example 8

### Purified 791Tgp72 Antigen

To confirm that the anti-CD55 mabs can bind to 791Tgp72, purified antigen (50 ng) or antigen released form PI-PLC treated 791T cells was added separately to flexible microtest plates (Falcon, Becton Dickinson, CA, USA) and left at 37° C. till dry. The plates were washed three times with phosphate buffered saline containing 0.05% Tween-20 (PBS-Tween) and blocked with BSA (1%) for 1 hr at room temperature. The plates were washed three times, then anti-CD55 antibodies (500 ng) in PBS were added. After 1 hr at room temperature the plates were washed three times in PBS-Tween and conjugated rabbit anti-mouse horseradish peroxidase (HRPO) diluted 1:1000 was added for a further 1 hr. Finally, after extensive washing, the plates were developed and read at 405 nm.

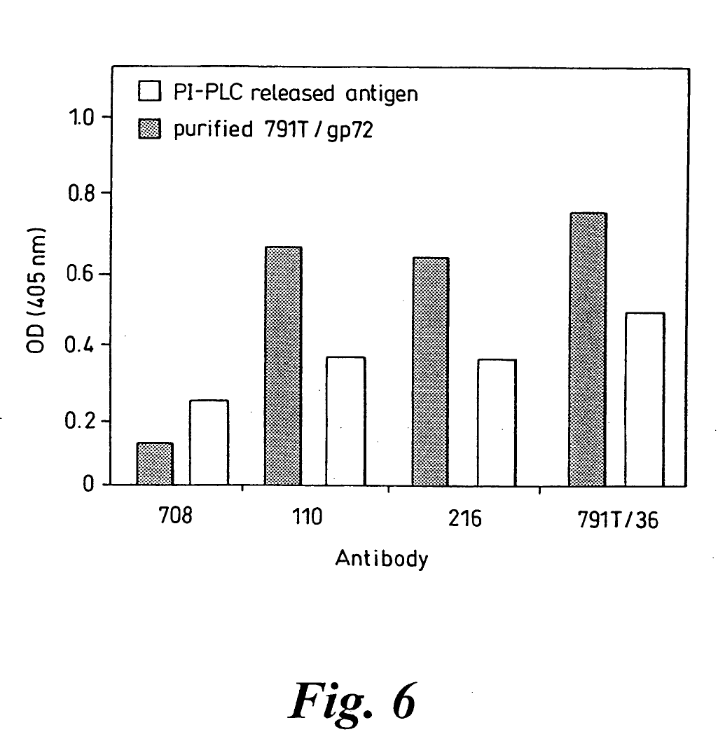

The binding of 791Tgp72 antigen to mAb 791T/36 and other anti-CD55 antibodies was shown by ELISA. The binding of both 791T/36 and anti-CD55 antibodies to purified 791Tgp72 antigen was clearly seen. 791T/36 also showed significant binding to 791Tgp72 antigen released from PI-PLC treated 791T cells (FIG. 6). The binding of the anti-CD55 mAbs to purified 791Tgp72 confirm that this antigen shares considerable homology with CD55.

### Example 9

### Mapping of the 791T/36 Epitope

DAF (CD55) consists of 4 SUSHI domains, a C-terminal O-glycosylated tail and a GPI anchor. Purified 791Tgp72 was used in a sandwich ELISA to determine to which domain 791T/36 bound. The antigen was captured with either one of the anti-CD55 Mabs or 791T/36 and then detected with 791T/36. Thus, recognition of the antigen by the same antibody as the capture antibody would indicate that the antibody is able to bind to two sites on the purified 791Tgp72. Conversely, absence of binding would indicate that the antibody has only one binding site on the antigen. In this way, antibodies can be mapped to the different domains of CD55. Plates were coated with anti-CD55 antibodies 220 (SUSHI domain 1), 110 (SUSHI domain 2), BRIC 216 (SUSHI domain 3) and left at 4° C. overnight. The plates were washed three times with PBS-Tween and blocked with BSA (1%) for 1 hr at room temperature. The plates were washed three times, then purified 791Tgp72 antigen (25ng) was added. After 1 hr at room temperature, the plates were washed three times and biotinylated mAb 791T/36 (500 ng per well) was added. Following incubation at room temperature for 1 hr, and washing three times, streptavidin-HRPO diluted 1:1000 was added for a further 1 hr. After a further six washes, the plates were developed and read at 405 nm.

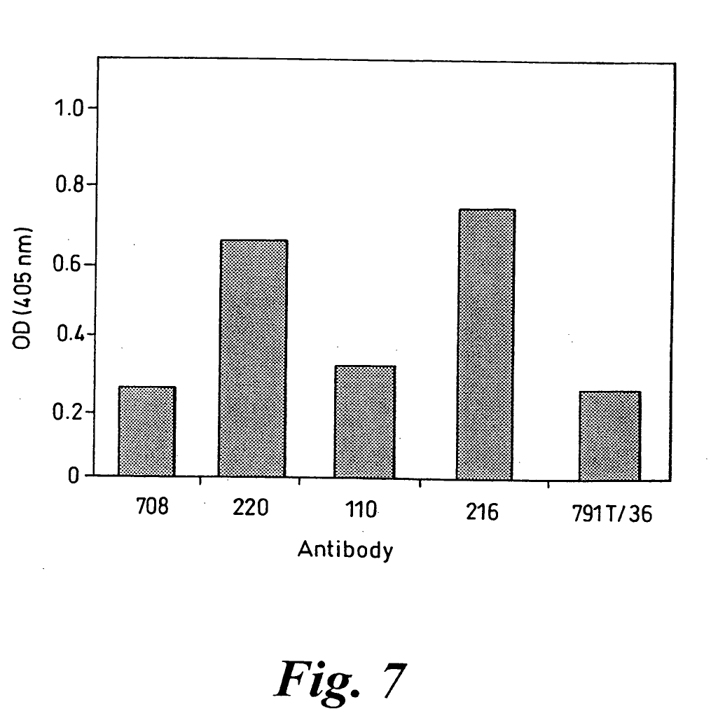

FIG. 7 shows that the 791Tgp72 antigen captured by monoclonal antibodies which bound to SUSHI domains 1 and 3 could be detected by 791T/36 biotin. Interestingly, capture of 791Tgp72 by mAb 110, which was raised against SUSHI domain 2, or 791T/36 could not be detected by 791T/36 biotin, suggesting that 791T/36 must bind near SUSHI domain 2.

The anti-CD55 antibodies were tested in a competition assay for their ability to inhibit the binding of 791T/36 to 791T cells. The inhibition of 791T/36 binding would indicate that the competing antibody bound to a similar or shared antigenic site on the 791Tgp72 molecule. 791T cells (2×105) were mixed with different amounts of cold anti-CD55 monoclonal antibodies at 37° C. for 30 minutes prior to adding mAb 791T/36 FITC (0.1 μg). After 1 hr at 37° C., the cells were washed two times with RPMI 1640 medium, fixed and measured by flow cytofluorometry.

Table 4 shows that in the competition binding assay of anti-CD55 with 791T/36 FITC, only cold mAb 791T/36 inhibited the binding of labelled 791T/36. These results suggest that although 791T/36 binds at or near SUSHI domain 2, it binds at a distinct site to monoclonal antibody 110.

### Example 10

### CHO Transfections with CD55/CD46 Chimeric Proteins

In order to ascertain the domain to which 791T/36 binds, a number of chimeric constructs were made comprising CD46, a membrane bound complement control protein with similar structure to CD55, i.e. contains four SUSHI domains but those domains are distinct to those of CD55. The constructs were produced by Dr Dale Cristiansen (Austen Research Centre, Victoria, Australia). The constructs tested were:
(1) CD46 (CD55 3); CD46 with SUSHI domain 3 substituted with that of CD55.
(2) CD46 (CD55 4); CD46 with SUSHI domain 4 susbtituted with that of CD55.
(3) CD46 (CD55 3/4); CD46 with SUSHI domains 3/4 substituted with those of CD55.
(4) CD46 (CD55 1/2); CD46 with SUSHI domains 1/2 substituted with those of CD55.

Only CHO cells transfected with constructs containing CD55 SUSHI2 showed significant binding to 791T/36 monoclonal antibody (Table 1).

### Example 11

### Anti-Idiotypic Antibodies

A human (105AD7) and a mouse (730) anti-idiotypic antibodies which bind at the antigen combining site of 791T/36 have been produced. A competition assay was used to assess if these anti-idiotypic antibodies could also bind to the other anti-CD55 antibodies. 791T cells (2×105) were mixed with anti-CD55 (0.1 μg) and varying amounts of 105AD7 or 730 at 37° C. for 1 hr. The cells were washed two times with RPMI 1640 medium prior to the addition of rabbit anti-mouse FITC (1:100) for a further 1 hr. The cells were washed two times with RPMI 1640 medium, fixed and measured by flow cytofluorometry.

The results in tables 5 and 6 indicate the binding of 791T/36 to 791T cells decreased when increasing concentration of mAb 105AD7 or 730 were added. In contrast, no loss in binding of the other anti-CD55 was seen in the presence of either anti-idiotype. These results add support to the conclusion that 791T/36 is a unique anti-CD55 monoclonal antibody.

The anti-idiotypic antibodies both stimulate humoral and cellular responses against cells which express 791Tgp72 antigen suggesting that they can mimic the antigen. Comparison of the amino acid sequences of both anti-idiotypes with CD55 show areas of homology with both CDRH3 regions of the antibodies and distinct regions of SUSHI domain 2. For 105AD7:
CDR L1- homology 7/9 amino acids with SUSHI1 83-93.
CDH H3- homology 5/7 amino acids with SUSHI2 151-158.
For 730:
CDR L1- homology 6/9 amino acids with SUSHI1 83-93.
CDH H3- homology 5/7 amino acids with SUSHI2 121-128.

### Example 12

### Western Blotting

To confirm that 791T/36 could immunoprecipitate CD55 from normal cells, erythrocytes (2×109) were washed two times with PBS and solubilized by NP-40 (1%, 10 ml). After centrifugation at 3000 rpm for 10 minutes, the supernatant was removed to a clean tube for centrifugation (100,000g×30 min). 10 μl of erythrocyte supernatant (equal to 2×106 of erythrocytes) and purified 791Tgp72 antigen (200 ng) was loaded onto SDS-PAGE at non-reducing condition as described previously. Proteins were transferred to nitrocellulose membrane and blocked with PBS containing BSA (1%) for 1 hr at room temperature (RT). After two times wash with PBS-Tween (0.1%), primary antibody was added for 1 hr at RT. The blots were washed two times and rabbit anti-mouse conjugate diluted 1:1000 was added. Following 1 hr incubation and extensive washing, the blots were developed by ECL system.

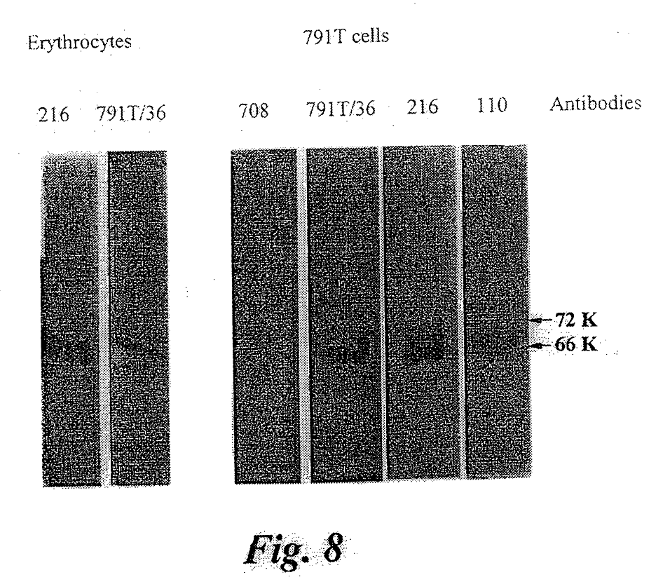

Detecting the 791Tgp72 antigen from erythrocytes and 791T cells by Western blotting indicated some differences only one band at 72 kDa was found on erythrocytes whereas two bands of 72 and 66 kDa exist on 791T cells (FIG. 8).

Treatment of the 72 kDa and 66 kDa bands with neuraminidase (which removes sialic acid residues from glycoproteins) yielded in each case a band of 55 kDa, suggesting that the 72 kDa and 66 kDa proteins are glycosylation variants of each other.

### Example 13

### Clone and DNA Sequence

To confirm the identity of 791Tgp72, the gene encoding this protein was cloned and sequenced. Total cellular RNA was isolated by the guanidine isothiocyanate method from 791T cells (4×107) grown in monolayers. First-strand CDNA synthesis was carried out using Ready-To-Go First-Strand Kit (Pharmacia Biotech, UK). Primers based on the N-terminal protein sequence obtained from the 72 and 66 KDa bands were generated;

| | |
|---|---|
| Pep 5′: | GACTGTGGCCTTCCCCCAG |
| C-CD55-5′: | AAAATGACCGTCGCGCGGCCG |
| C-CD55-3′: | CTAAGTCAGCAAGCCCATGGT |
| B-CD55-5′: | GAATACTGCAGATGACCGTCGCGCGGCCG |
| B-CD55-3′: | CCTACGAATTCTAAGTCAGCAAGCCCATGG |
| FL-CD55-3′: | ATGTGATTCCAGGACTGCC |
| FL-CD55-5′: | TGGGCGTAGCTGCGACTCG |

These primers were designed for the following:

C-CD55: Cloning and expression in eukaryotic cells from the recognised start codon to the stop codon of native CD55.

B-CD55: Cloning and expression into a bacterial expression vector in order to generate protein for purification. The sequences include the addition of a 5′ EcoRI site and a 3′ PstI site.

FL-CD55: were designed for cloning of the recognised coding region of CD55 and 200 bp of the 3′ untranslated region. This should allow the cloning of potential splice variants that occur in the 3′ end of the antigen. 791Tgp72 PCRs were set up with first strand cDNA, the primers used were mixes of the primer sets outlined above. The samples were placed in a thermal cycler, the following profile was used (hot start at 94° C. for 2 minutes; denaturation at 94° C. for 30 seconds, 55° C. for 45 seconds, 72° C. for 90 seconds, repeat for a total 30 cycles). PCR products were cloned into modified pBluescript SK-vector. Positive clones were checked by PCR using vector specific primers and the positive DNA plasmids sequenced on an ABI automated sequencer.

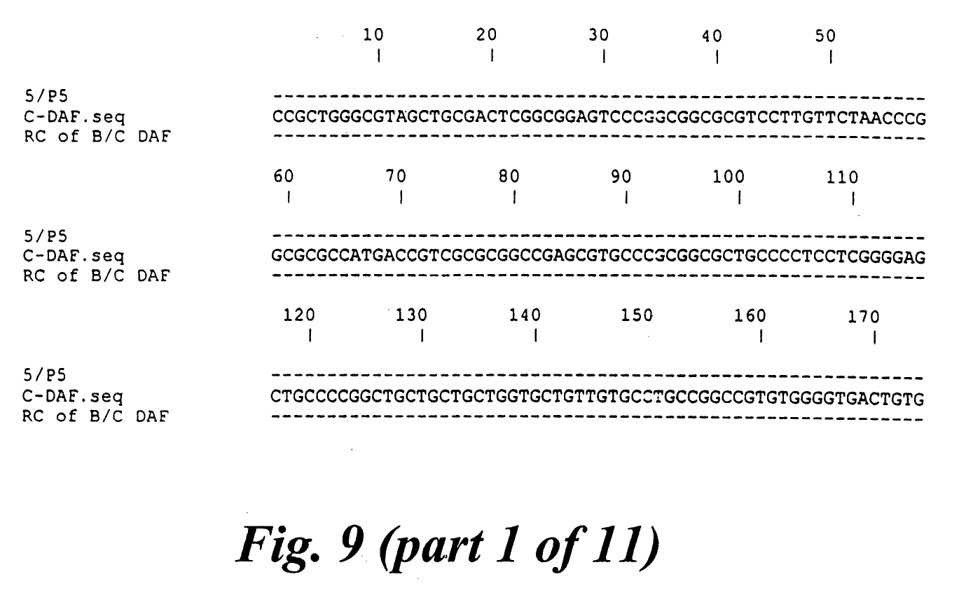

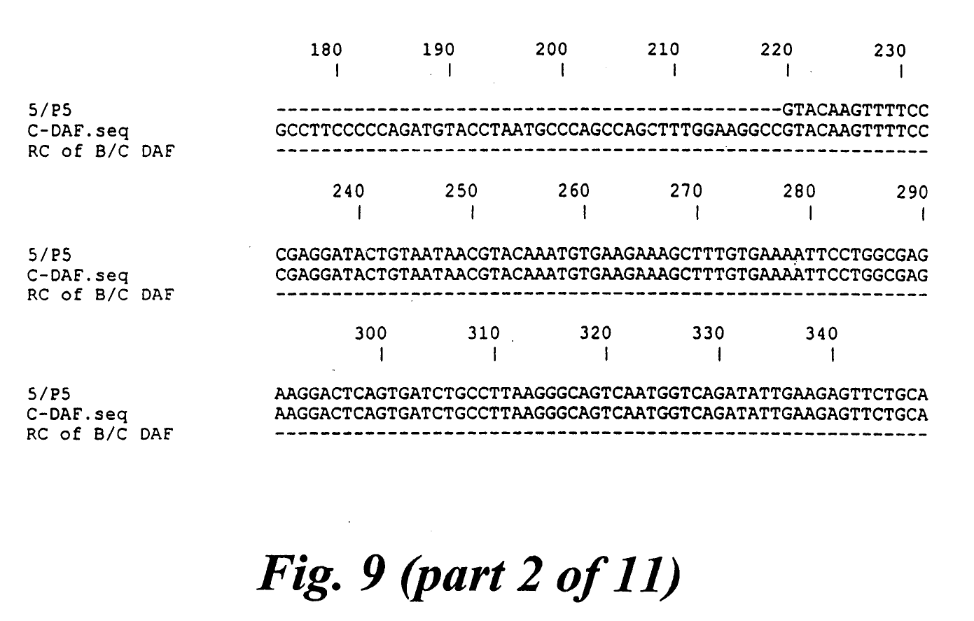

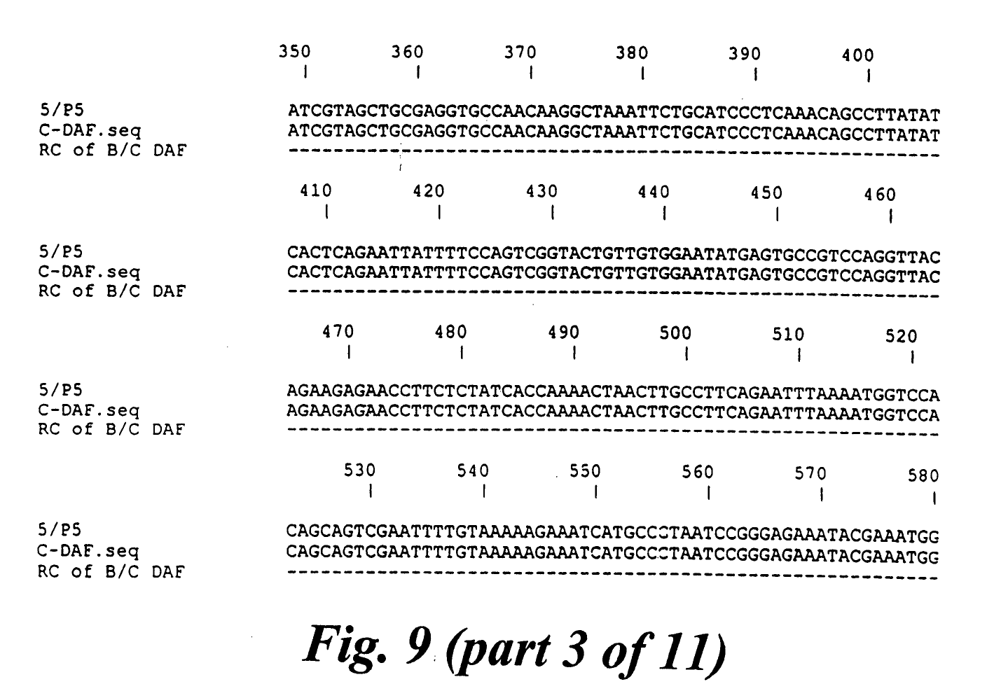

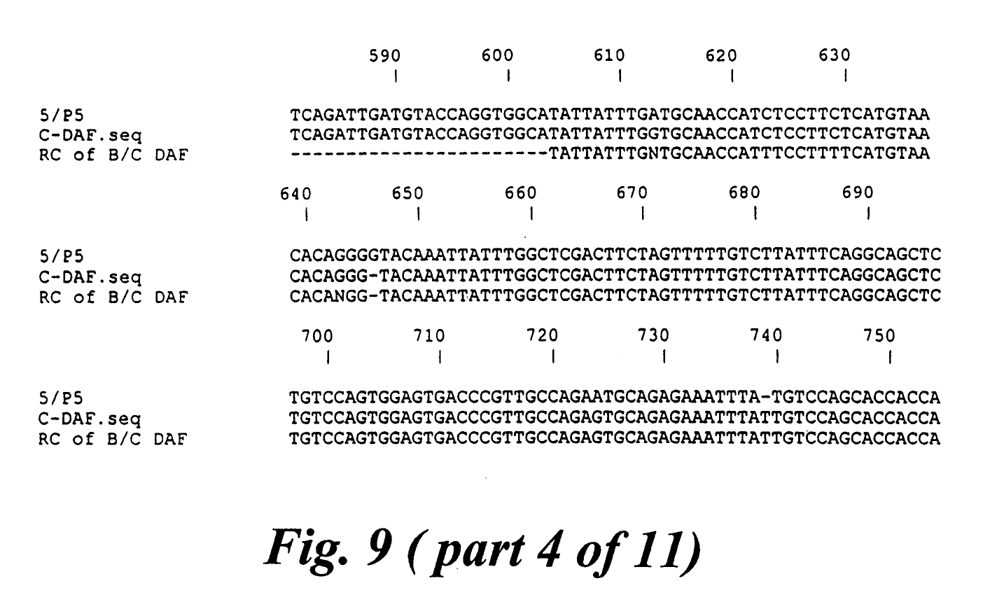

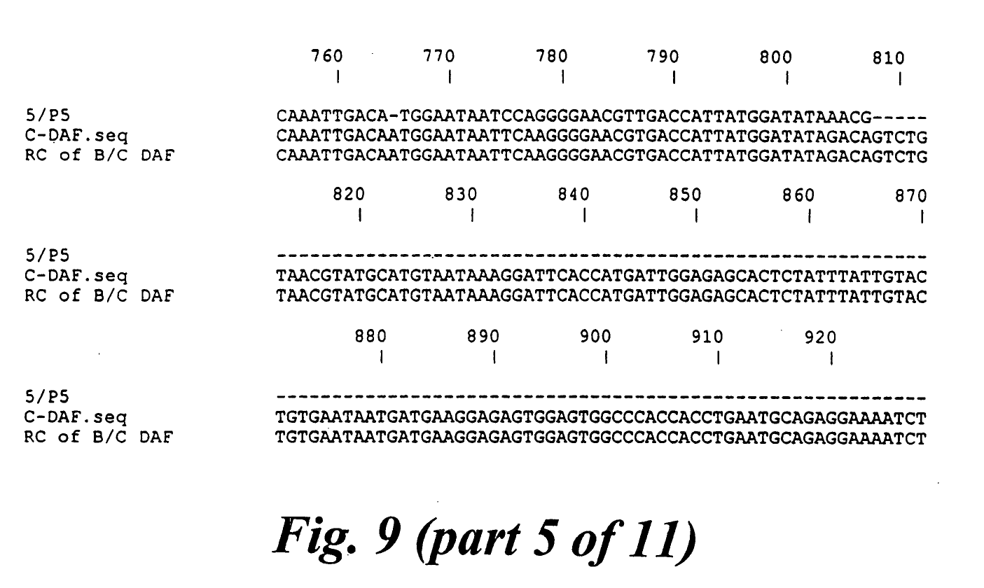

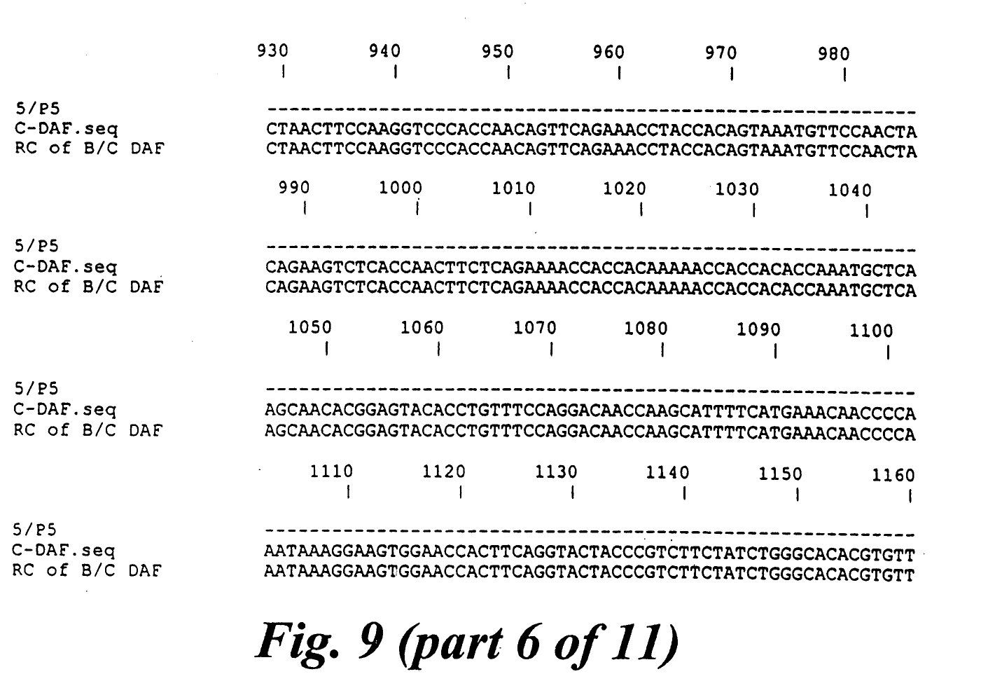

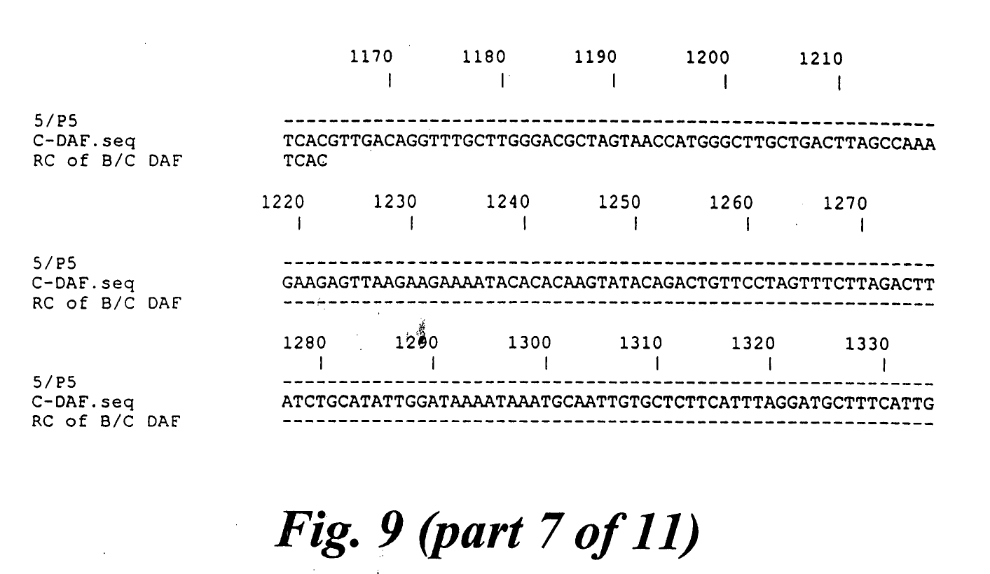

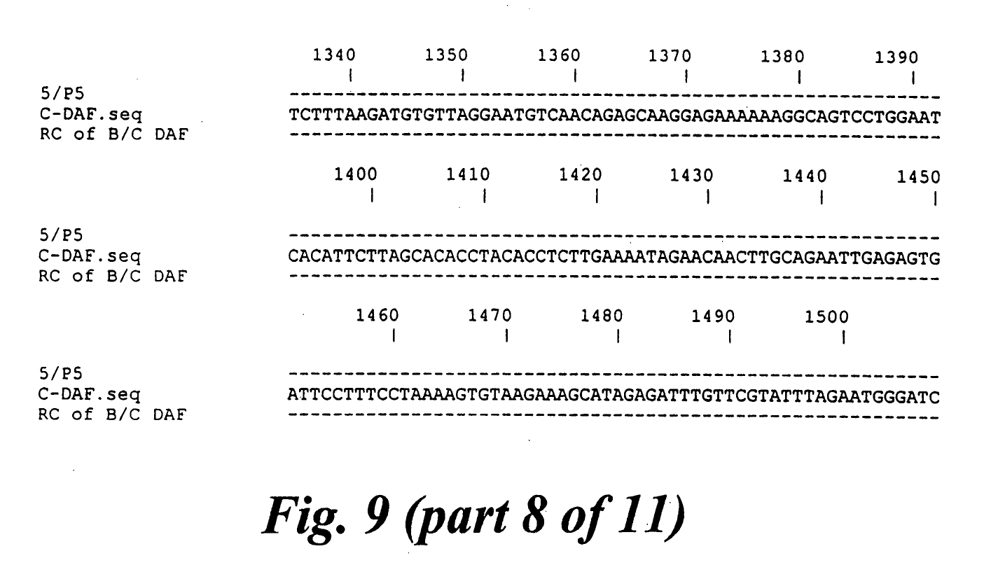

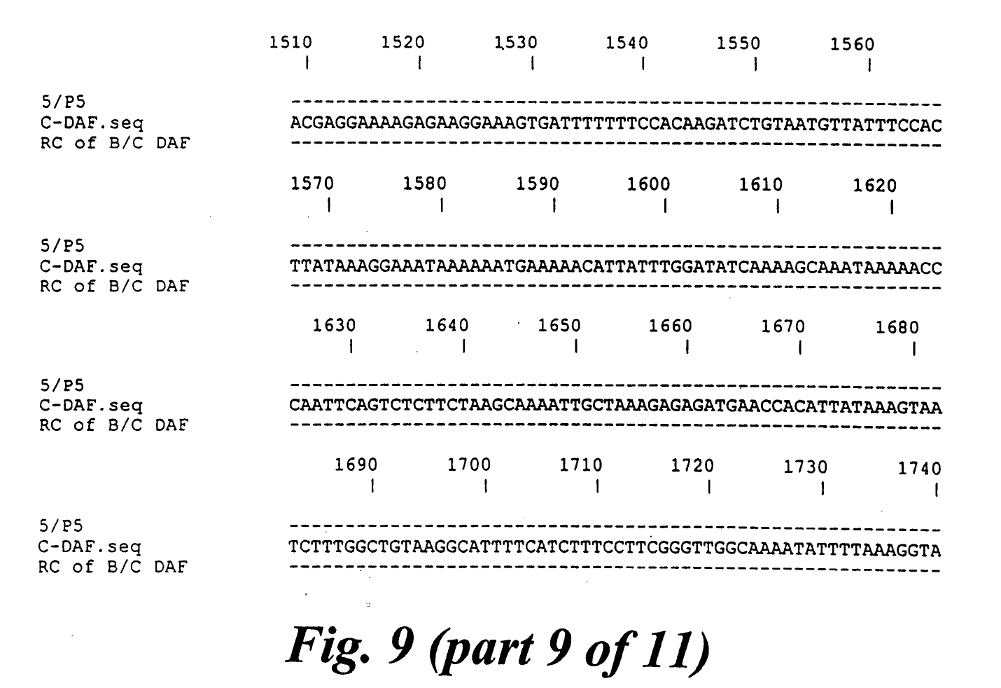

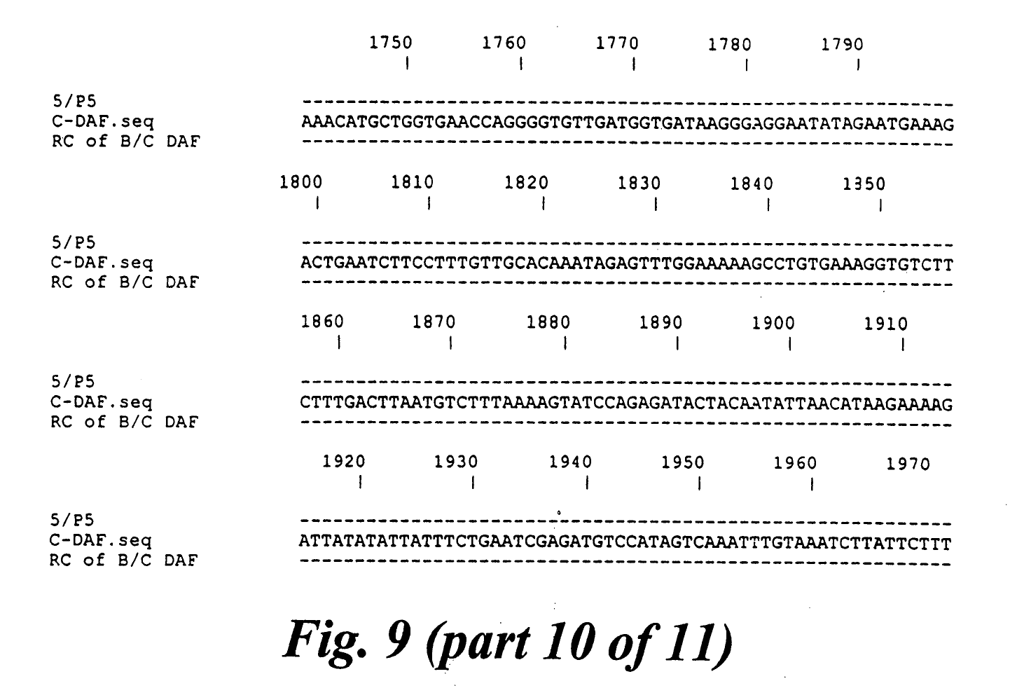

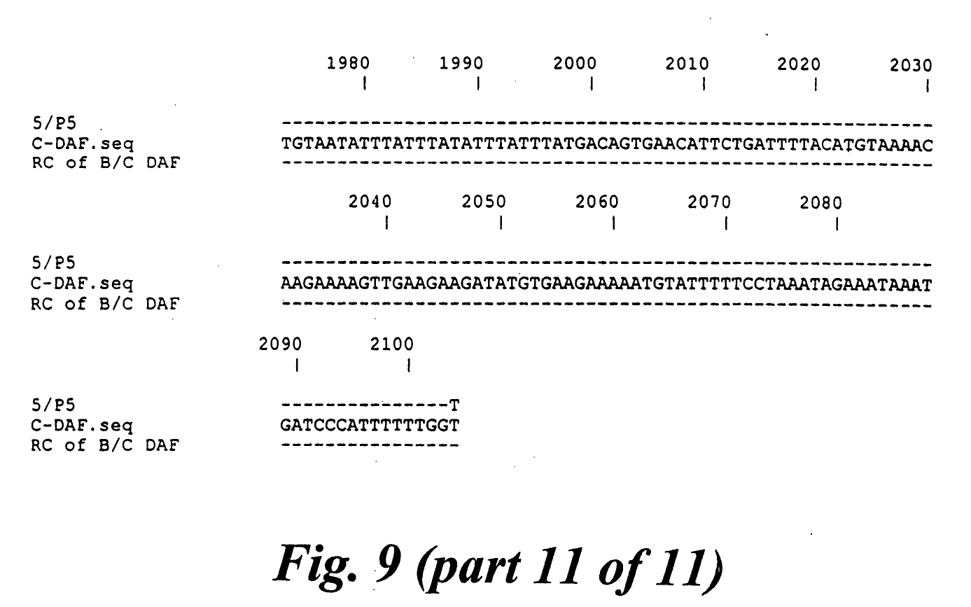

The initial cloning experiments resulted in products generated from PEP5′ and either CCD553′, BCD553, or FLCD553′. The results of this sequencing revealed there to be no difference in sequence between the cloned products and the full reported sequences of CD55 (FIG. 9). The translated amino acid sequence of CD55 is set out in FIG. 10.

Recently full length versions of the PCR generated CD55 products have been cloned using FLCD555′ and either FLCD553′, CCD553′or BCD553′.

Discussion

Interest in the use of 791Tgp72 as a target for immunotherapy arose initially by the demonstration that this antigen was expressed by the majority of osteosarcomas, colorectal, gastric and ovarian tumours. The tumour specificity of 791Tgp72 was also emphasised by extensive clinical imaging studies with radiolabelled anti-791Tgp72 monoclonal antibody 791T/36, in the detection of primary and metastatic colorectal cancer, osteosarcoma, breast and ovarian cancer. The results shown herein relate to the first isolation of 791Tgp72 and the use of this antigen or a related family member CD55 as a cancer vaccine.

From the prior art, CD55 is a very surprising target for T-cell immunity as it is expressed on essentially all haematopoietic cells and on endothelial and epithelial tissues, including the vascular endothelium, gastrointestinal tract, genitourinary tract, central nervous system, and extracellular matrix. 791T/36 binds weakly to erythrocytes and it may be that this has been advantageous in the clinical imaging studies. 791T/36 antibody may have bound weakly to erythrocytes which upon passage through the tumour have allowed transfer of the antibody to 791Tgp72 to which it binds with higher affinity. CD55 was initially purified based on its ability to accelerate the decay of the classical pathway C3 convertase, C4b2a. It carries out the same function with respect to the alternative pathway C3 convertase, C3bBb, but does not have any cofactor activity for the factor I-mediated proteolytic degradation of C3b or C4b. So CD55 protects the cell from complement-mediated lysis at the C3 convertase step.

Normal human tissues express membrane-associated complement inhibitory proteins that protect these tissues from damage by autologous complement. To determine whether neoplasms also express these proteins prior investigators have examined the distribution of CD55 (DAF), CD59 (protectin) and CD46 (membrane cofactor protein) in frozen samples of human breast, colon, kidney, and lung carcinomas and in adjacent non-neoplastic tissues. Difference between normal tissues and the corresponding neoplasms were observed, with loss or gain of expression of one or more inhibitors. Some tumours expressed only one inhibitor whether others expressed different combinations of two or three inhibitors. Colon carcinomas, by contrast, expressed all inhibitors. The results demonstrate that most carcinomas, with the exception of small cell carcinomas of the lung, do express one or more complement inhibitors at a level likely to inhibit complement-mediated cellular damage. Other tumour tissues, such as ovarian and gastrointestinal tumour cells, were also checked. The surface expression level of CD55 varied, and correlation with the vulnerability of the cells to C-mediated lysis. Thus, the expression of C regulators on malignant cells may constitute a tumour escape mechanism, and is a critical parameter to be examined when mAb therapy is being considered. Furthermore, expression of CD55 on target cells makes them resistant to lysis by natural killer cells. Many tumours escape T-cell recognition by loss of MHC molecules, however this makes them susceptible to NK killing. Over-expression of CD55 which inhibits NK lysis is therefore an obvious advantage.

The extensive expression of CD55 on normal cells, its role in protecting cells from complement and NK lysis makes a very unlikely target for T-cell immunotherapy. However, clinical trials with 105AD7 which mimics an epitope on CD55 are showing that it can stimulate excellent T-cell responses. 791Tgp72 does however show some differences from CD55. Two bands are precipitated from tumour cells whereas only one band is seen in erythrocytes. Although the -anti-CD55 antibodies can precipitate the 791Tgp72 from tumour cells the yield is much lower than is observed with 791T/36. This is reflected in the cell binding assays where 791T/36 shows strongest binding to tumour cells whereas the anti-CD55 monoclonal antibody BRIC 216 binds better to. erythrocytes. Different forms of CD55 have been isolated from tissue such as erythrocytes, urine and tears (Nakano et al, 1991; Sugita et al, 1988; Seya et al, 1995). CD55-A (63kDa) and CD55-B (55kDa) from erythrocytes do not appear to have a GPI anchor. CD55-U2 (60-80kDa) in urine is thought to be inactive. The existence of a human splice variant of CD55 has been suggested but the putative protein has never been isolated. Furthermore, new functions other than complement decay have been suggested. Activated T-cells which have been crosslinked with anti-CD55 monoclonal antibodies can induce T-cell proliferation and signal transduction. It is unclear if this is related to the recent observation that CD55 is the ligand for the CD97 receptor expressed on activated T-cells.

Whether there are different roles for CD55 or different forms of CD55/791Tgp72 in tumour cells or whether there is differential it remains an interesting prospect to use a molecule which tumours over-express to protect themselves from immune attack as a cancer vaccine. The dichotomy being that if the cell fails to express the molecule it is susceptible to complement mediated and NK lysis and if it does express the antigen it will be killed by CD55 specific T-cells.

### References

The references mentioned herein are all expressly incorporated by reference.
Embleton et al, Br.J. Cancer 1981; 43: 582-587.
Price et al, Br.J. Cancer 1984; 49: 809-812.
Durrant et al, Cancer Res. 1986; 46: 3543-3549.
Durrant et al, J.Natl. Cancer Inst. 1989; 81: 688-695.
Durrant et al, British Journal Of Cancer 1989; 60: 855-860.
Durrant et al, Clinical and Experimental Immunology 1989; 75: 258-264.
Austin et al, Immunol 1989; 67: In press.
Altin et al, Immunol., Cell Biol. 1994; 72: 87-96.
Laemmli, Nature 1970; 227: 680-685.
Stern, Immunocytochemistry of Embryonic Material. Oxford: IRL press, 1993.
Dunbar, Protein Blotting: A Practical Approach. Oxford: IRL Press, 1994.
Hole et al, Br. J. Cancer 1988; 57: 239-246.
Hole et al, Int.J. Cancer 1990; 45: 179-184.
Goding, Monoclonal antibody: Principles and Practice.
London: Academic Press, 1996.
Schneider et al, J. Biol. Chem. 1982; 257: 10766-10769.
Nicholson-Weller et al, J.Immunol., 1981; 127: 2035-2039.
Nicholson-Weller et al, J. Immunol., 1982; 129: 184-189.
Seya et al, International Immunology 1995; 7: 727-736.
Nakano et al, Biochem. Biophys. Acta. 1991; 1074: 326-330.

Sugita et al, J. Biochem. 1988; 104: 633-637.

TABLE 1
Fluorescence of Mabs on chimeric transfectants

| Constructs | 791T/36 | 1H4 | E4.3 |
|---|---|---|---|
| wt DAF | 105 | 164 | 16 |
| DAF 3 | 4 | 94 | 79 |
| DAF 4 | 3 | — | 70 |
| DAF 3/4 | 5 | — | 70 |
| DAF 1/2 | 108 | — | 3 |

(a) 1H4 Mab binds to sushi domain 3 of DAF
(b) E4.3 Mab binds to sushi domain 1 of CD 46
Chimeric constructs were transfected into CHO cells and assayed for their binding of 791T/36, 1H4 and E4.3.

TABLE 2
Indirect Immunofluorescence Assay Of Various Antibodies
On DAF Positive And Negative Cells
Mean Linear Fluorescence

| Antibody | Erythrocytes | 791T | HUVEC |
|---|---|---|---|
| 110 | 52.71 | 1186.96 | 109.33 |
| 216 | 94.28 | 1776.41 | 172.52 |
| 791T/36 | 60.07 | 2373.89 | 146.96 |
| Rabbit anti- | 26.68 | 27.88 | 55.55 |
| mouse FITC | | | |
| 708 | 24.11 | 36.42 | 47.38 |

Monoclonal antibodies to DAF (110, 216) and 791Tgp72 (791T36) were used to label a range of cells for 1 hour at 4° C. Cells were then incubated with FITC-labelled rabbit anti mouse antibody and read by FACS. Analysis was also carried out using Mab 708 as a negative control and with Rabbit anti-mouse FITC alone.

TABLE 3
Binding Of 791T/36, Control IgG2b (708) And Anti-DAF
Antibodies To 791T Cells Following
Treatment With PI-PLC At 1 u/ml For 1 Hour.

| Antibodies | PI-PLC (−) | PI-PLC(+) | % Inhibition |
|---|---|---|---|
| 110 | 963.40 | 86.34 | 91 |
| 216 | 1101.51 | 208.83 | 82 |
| 791T/36 | 1991.35 | 178.6 | 91 |
| 708 | 29.79 | 25.1 | 14 |

Mean linear fluorescence readings of anti DAF (110, 216) Mabs and anti 791T/gp72 (791T/36) antibodies to phospholipase treated or untreated 791T cells. 708 Mab was used as a control antibody.

TABLE 4
Competitive Binding Of 791T/36 FITC (0.1 ug)
To 791T Cells By Anti DAF Antibodies,
Unlabelled 791T/36 Or An Irrelevant IgG2b Mab
Which Does Not Bind To 791T Cells

| Cold | | | | | | |
|---|---|---|---|---|---|---|
| Mab | | | | | | % Inhibition |
| (ug) | 708 | 220 | 110 | 216 | 791T/36 | with 791T/36 |
| 0 | 453.82 | 450.96 | 459.65 | 502.11 | 473.21 | 0 |
| 0.1 | 426.73 | 505.04 | 488.02 | 456.44 | 335.41 | 30 |
| 0.5 | 451.60 | 499.91 | 493.05 | 496.46 | 162.38 | 66 |
| 1.0 | 454.20 | 455.08 | 497.82 | 509.80 | 103.47 | 79 |

FITC labelled 791T/36 antibody was incubated with various concentrations of the above unlabelled antibodies. Only unlabelled 791T/36 was able to inhibit binding of the FITC labelled 791T/36. Mean linear fluorescence readings are given.

TABLE 5
Competition Assay Of 105AD7 With Various
Anti-DAF Antibodies On 791T Cell

| 105AD7 (ug) | 708 | 110 | 216 | 791T/36 |
|---|---|---|---|---|
| 0 | 15.03 | 489.87 | 803.19 | 912.46 |
| 0.05 | 14.84 | 528.65 | 701.71 | 1089.17 |
| 0.1 | 17.24 | 388.11 | 783.55 | 912.35 |
| 0.5 | 14.72 | 533.11 | 607.61 | 726.25 |
| 1.0 | 16.14 | 512.53 | 626.31 | 570.13 |
| 5.0 | 20.85 | 370.53 | 562.24 | 53.07 |
| Anti-DAF antibodies (110, 216) and anti 791T/gp72 (791T/36) were added to 791T cells in the presence of increasing concentrations of 105AD7 anti-idiotypic antibody, which specifically recognises the binding site of 791T/36. Cells were incubated for 1 hour at 37° C. then for a further hour in the presence of FITC-labelled Rabbit anti-mouse. Cells were analysed by FACS. 708 Mab was used as a negative control. The results indicate that only 791T/36 was inhibited by 105AD7. Mean linear | | | | |
| # fluorescence readings are given. | | | | |

TABLE 6
Competition Assay Of 730 With Various
Anti-DAF Antibodies On 791T Cell

| 730 (ug) | 708 | 110 | 216 | 791T/36 |
|---|---|---|---|---|
| 0 | 19.97 | 607 | 1356 | 1382 |
| 0.1 | 17.63 | 642 | 1185 | 1352 |
| 0.5 | 19.02 | 632 | 1158 | 983 |
| 1.0 | 19.31 | 620 | 1229 | 597 |
| 2.5 | 23.10 | 739 | 1212 | 89 |
| 5.0 | 32.54 | 640 | 1179 | 70 |
| Anti-DAF antibodies (110, 216) and anti 791T/gp72 (791T/36) were added to 791T cells in the presence of increasing concentrations of Mab 730 anti-idiotypic antibody, which specifically recognises the binding site of 791T/36. Cells were incubated for 1 hour at 37° C. then for a further hour in the presence of FITC-labelled Rabbit anti-mouse. Cells were analysed by FACS. 708 Mab was used as a negative control. The results indicate that only 791T/36 was inhibited by 730. | | | | |
| # Mean linear fluorescence readings are given. | | | | |

1-26. (canceled)

27. A method of inducing an immune response in a cancer patient, the method comprising administering to said cancer patient a polypeptide, wherein the polypeptide is either:
(a) a fragment of the polypeptide whose amino acid sequence is shown in FIG. 10, the fragment comprising amino acids 97-159 of FIG. 10; or
(b) a derivative of said fragment having 25 or fewer amino acid insertions, additions, deletions and/or substitutions compared to the fragment, and wherein the polypeptide is optionally linked to a peptidyl or non-peptidyl coupling partner, and is capable of inducing an immune response in a cancer patient.

28. The method of claim 1, wherein the polypeptide has at least five amino acids identical with corresponding amino acids of a contiguous stretch of seven amino acids contained within amino acids 121-128 or 151-158 of FIG. 10.

29. The method of claim 1, wherein the polypeptide has at least six amino acids identical with corresponding amino acids of a contiguous stretch of nine amino acids contained within amino acids 83-93 of FIG. 10.

30. The method according to claim 1, wherein the derivative has 15 or fewer amino acid insertions, additions, deletions and/or substitutions compared to the fragment.

31. The method according to claim 1, wherein the derivative has 10 or fewer amino acid insertions, additions, deletions and/or substitutions compared to the fragment,

32. The method according to claim 1, wherein the derivative has 5 or fewer amino acid insertions, additions, deletions and/or substitutions compared to the fragment.


SEQUENCE LISTING (13 sequences)

SEQ ID NO: 1 - DNA, 2102 bp, Homo sapiens
  CDS (66)..(1211)
ccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctacacctcttgaaaatagaacaacttgcagaattgagagtgattcctttcctaaaagtgtaagaaagcatagagatttgttcgtatttagaatgggatcacgaggaaaagagaaggaaagtgatttttttccacaagatctgtaatgttatttccacttataaaggaaataaaaaatgaaaaacattatttggatatcaaaagcaaataaaaacccaattcagtctcttctaagcaaaattgctaaagagagatgaaccacattataaagtaatctttggctgtaaggcattttcatctttccttcgggttggcaaaatattttaaaggtaaaacatgctggtgaaccaggggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggt

SEQ ID NO: 2 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 3 - DNA, 586 bp, Homo sapiens
gtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttgatgcaaccatctccttctcatgtaacacaggggtacaaattatttggctcgacttctagtttttgtcttatttcaggcagctctgtccagtggagtgacccgttgccagaatgcagagaaatttatgtccagcaccaccacaaattgacatggaataatccaggggaacgttgaccattatggatataaacgt

SEQ ID NO: 4 - DNA, 559 bp, Homo sapiens
  misc_feature (10, 39): n is a or g or c or t
tattatttgntgcaaccatttccttttcatgtaacacanggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcac

SEQ ID NO: 5 - DNA, 1493 bp, Homo sapiens
  CDS (199)..(1344)
tttaaacgggccctctagactcgagcggccgctgcccatcttgtcgtcgtcgtccttgtagtcgtgcatgtggtggtggtggtggtggttaaccatggtggcgggccgccactgtgctggatatctgcagaattcgatgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaaca

SEQ ID NO: 6 - protein, 16 aa, Homo sapiens
DCGLPPDVPNAQPALE

SEQ ID NO: 7 - DNA, 19 bp, Artificial Sequence
  Description of Artificial Sequence Primer
gactgtggccttcccccag

SEQ ID NO: 8 - DNA, 21 bp, Artificial Sequence
  Description of Artificial Sequence Primer
aaaatgaccgtcgcgcggccg

SEQ ID NO: 9 - DNA, 21 bp, Artificial Sequence
  Description of Artificial Sequence Primer
ctaagtcagcaagcccatggt

SEQ ID NO: 10 - DNA, 29 bp, Artificial Sequence
  Description of Artificial Sequence Primer
gaatactgcagatgaccgtcgcgcggccg

SEQ ID NO: 11 - DNA, 30 bp, Artificial Sequence
  Description of Artificial Sequence Primer
cctacgaattctaagtcagcaagcccatgg

SEQ ID NO: 12 - DNA, 19 bp, Artificial Sequence
  Description of Artificial Sequence Primer
atgtgattccaggactgcc

SEQ ID NO: 13 - DNA, 19 bp, Artificial Sequence
  Description of Artificial Sequence Primer
tgggcgtagctgcgactcg